SEQUENCE LISTING (52 sequences)

SEQ ID NO: 1 - DNA, 924 bp, Mouse
atgtggcagttttgcattctgtgcctctgtgtactcatggcttctgtggctacagaccccacagtgactttgctggagcaaaacccaaggtggcgtctggtaccacgtggtcaagctgtgaacctacgctgcatcttgaagaattcccagtatccctggatgagctggtatcagcaggatctccaaaagcaactacagtggctgttcactctgcggagtcctggggacaaagaggtcaaatctcttcccggtgctgattacctggccacacgggtcactgatacggagctgaggctgcaagtggccaacatgagccagggcagaaccttgtactgcacctgcagtgcatactggggtggaaacaccttgtactttggtgcgggcacccgactatcggtgctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 2 - protein, 307 aa, Mouse
MWQFCILCLCVLMASVATDPTVTLLEQNPRWRLVPRGQAVNLRCILKNSQYPWMSWYQQDLQKQLQWLFTLRSPGDKEVKSLPGADYLATRVTDTELRLQVANMSQGRTLYCTCSAYWGGNTLYFGAGTRLSVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 3 - DNA, 20 bp, Artificial Sequence
  Description of Artificial Sequence  Primer
gcgctcaggatgcataaaag

SEQ ID NO: 4 - DNA, 24 bp, Artificial Sequence
  Description of Artificial Sequence  Primer
ccccacagagatagagagaacctg

SEQ ID NO: 5 - protein, 7 aa, Mouse
RTLYCTC

SEQ ID NO: 6 - protein, 10 aa, Mouse
SAYWGGNTLY

SEQ ID NO: 7 - protein, 10 aa, Mouse
FGAGTRLSVL

SEQ ID NO: 8 - protein, 8 aa, Mouse
VSRPGSGG

SEQ ID NO: 9 - protein, 15 aa, Mouse
KLTLGLEQDFRSTLT

SEQ ID NO: 10 - protein, 10 aa, Mouse
SRTQNLLCTS

SEQ ID NO: 11 - protein, 7 aa, Mouse
SVYFCAS

SEQ ID NO: 12 - protein, 9 aa, Mouse
GWGGSYEQY

SEQ ID NO: 13 - protein, 10 aa, Mouse
FGPGTRLTVL

SEQ ID NO: 14 - protein, 6 aa, Mouse
LSNWDN

SEQ ID NO: 15 - protein, 18 aa, Mouse
TGYQNFYFGKGTSLTVIP

SEQ ID NO: 16 - protein, 10 aa, Mouse
NIQNPEPAVY

SEQ ID NO: 17 - protein, 7 aa, Mouse
SLYFCAS

SEQ ID NO: 18 - protein, 9 aa, Mouse
SDGGNSDYT

SEQ ID NO: 19 - protein, 10 aa, Mouse
FGSGTRLLVI

SEQ ID NO: 20 - protein, 7 aa, Mouse
SVYFCAS

SEQ ID NO: 21 - protein, 9 aa, Mouse
SDALFYEQY

SEQ ID NO: 22 - protein, 10 aa, Mouse
FGPGTRLTVL

SEQ ID NO: 23 - protein, 7 aa, Mouse
SVYFCAS

SEQ ID NO: 24 - protein, 10 aa, Mouse
GDAQANERLF

SEQ ID NO: 25 - protein, 10 aa, Mouse
FGHGTKLSVL

SEQ ID NO: 26 - protein, 7 aa, Mouse
SVYFCAS

SEQ ID NO: 27 - protein, 9 aa, Mouse
GDNSAETLY

SEQ ID NO: 28 - protein, 10 aa, Mouse
FGSGTRLTVL

SEQ ID NO: 29 - protein, 7 aa, Mouse
RTLYCTC

SEQ ID NO: 30 - protein, 10 aa, Mouse
SAYWGGNTLY

SEQ ID NO: 31 - protein, 10 aa, Mouse
FGAGTRLSVL

SEQ ID NO: 32 - protein, 7 aa, Mouse
SVYFCAS

SEQ ID NO: 33 - protein, 10 aa, Mouse
GDRGSQDTQY

SEQ ID NO: 34 - protein, 10 aa, Mouse
FGPGTRLLVL

SEQ ID NO: 35 - protein, 7 aa, Mouse
ATYLCAS

SEQ ID NO: 36 - protein, 6 aa, Mouse
SPYEQY

SEQ ID NO: 37 - protein, 10 aa, Mouse
FGPGTRLTVL

SEQ ID NO: 38 - protein, 7 aa, Mouse
SLYFCAS

SEQ ID NO: 39 - protein, 8 aa, Mouse
RDSQNTLY

SEQ ID NO: 40 - protein, 10 aa, Mouse
FGAGTRLSVL

SEQ ID NO: 41 - protein, 7 aa, Mouse
SVYFCAS

SEQ ID NO: 42 - protein, 10 aa, Mouse
GEPLGAGEQY

SEQ ID NO: 43 - protein, 10 aa, Mouse
FGPGTRLTVL

SEQ ID NO: 44 - protein, 7 aa, Mouse
SLYFCAS

SEQ ID NO: 45 - protein, 10 aa, Mouse
RSGTTNSDYT

SEQ ID NO: 46 - protein, 10 aa, Mouse
FGSGTRLLVI

SEQ ID NO: 47 - protein, 7 aa, Mouse
SVYFCAS

SEQ ID NO: 48 - protein, 7 aa, Mouse
GANTEVF

SEQ ID NO: 49 - protein, 10 aa, Mouse
FGKGTRLTVV

SEQ ID NO: 50 - protein, 7 aa, Mouse
RTLYCTC

SEQ ID NO: 51 - protein, 7 aa, Mouse
RAYYEQY

SEQ ID NO: 52 - protein, 10 aa, Mouse
FGPGTRLTVL

(19) United States
(12) Patent Application Publication
(10) Pub. No.: US 2006/0127377 A1
(43) Pub. Date: Jun. 15, 2006

(54) T cell receptors with enhanced sensitivity recognition of antigen

(76) Inventors: Stanislav Vukmanovic, Kensington, MD (US); Fabio R. Santori, Edison, NJ (US); Zoran Popmihajlov, New York, NY (US)

Correspondence Address: Michael L. Goldman;Nixon Peabody LLP, Clinton Square, P.O. Box 31051, Rochester, NY, 14603-1051, US

(21) Appl. No.: 11/194,879

(22) Filed: Aug. 1, 2005

Related U.S. Application Data
(60) Provisional application No. 60/598,383, filed on Aug. 3, 2004.

(51) Int. Cl. A61K 48/00; C12N 5/08

(52) U.S. Cl. 424/93.21; 435/372

(57) ABSTRACT

The present invention relates to methods of treating an infectious, proliferative, or lymphocyte-mediated disease that involve providing a T cell receptor β chain (TCR β) having higher sensitivity recognition of antigen than a wild type TCR β chain and introducing the TCR β chain directly or indirectly to a subject having the disease under conditions effective to treat the disease. Also provided is another method of treating such diseases that involves providing an isolated mouse TCR β chain having higher sensitivity recognition of antigen than a wild type TCR β chain, linking the mouse TCR β chain with a human TCR αchain, and introducing the linked mouse TCR β and human TCR α chains to a subject having a disease, thereby treating the disease. The present invention also relates to a transgenic mouse having a TCR β chain having higher sensitivity recognition of antigen than a wild type mouse.

This application claims benefit of U.S. Provisional Patent Application Ser. No. 60/598,383, filed Aug. 3, 2004, which is hereby incorporated by reference in its entirety.

This invention was developed with government funding under National Institutes of Health Grant Nos. RO1 A1 41573 and RO1 A1 48837. The U.S. Government may retain certain rights.

### FIELD OF THE INVENTION

The present invention relates to a T cell receptor with enhanced sensitivity recognition of antigen and uses thereof for the treatment of proliferative, infectious, and lymphocyte-mediated diseases.

### BACKGROUND OF THE INVENTION

The vertebrate immune system is a complex defense system that has evolved to provide protection from the invasion of pathogenic microorganisms and cancer. One of the important features of this defense system is the type of white blood cells known as T lymphocytes (T cells). The protective function of T cells depends on their ability for antigen recognition, that is, the ability to recognize cells that are harboring pathogens or that have internalized pathogens or pathogen products. T cells do this by recognizing peptide fragments of pathogen-derived proteins bound to major histocompatibility complex (MHC) molecules on the surface of infected cells. MHC molecules are cell-surface glycoproteins with a peptide-binding groove that can bind a wide variety of different peptides. An MHC molecule binds a peptide in an intracellular location and delivers it to the cell surface, where the combined ligand can be recognized by a T cell. There are two classes of MHC molecules, MHC class I and MHC class II, which bind peptides from proteins degraded in different intracellular sites. MHC class I molecules bind peptides from proteins degraded in the cytosol. MHC II binds proteins that are degraded in endosomes.

Different types of T cells are activated on recognizing foreign peptides presented by the different classes of MHC molecules. T cell subpopulations can be distinguished by the presence of one or the other of two membrane molecules, CD4 and CD8, on their cell surface. T cells that express CD8 (CD8⁺ T cells) recognize MHC I:peptide complexes, and are specialized to kill cells displaying foreign peptides, thereby protecting the body from viruses and other pathogens that invade the cytosol. T cells that express CD4 (CD4⁺ T cells) recognize antigen associated with MHC class II cells, and are specialized to activate other immune responses in the cell.

Antigen recognition by CD8⁺ T cells plays a crucial role in adaptive immunity to infectious agents and tumors. The T cell receptors (TCRs) on the cell surface of CD8⁺ T cells recognize short peptide fragments presented by MHC class I molecules. Structurally, the MHC class I molecule is a membrane-bound, 44-kD heterotrimeric complex comprised of a heavy chain composed of an α1/α2 outer domain and a proximal α3 domain, a β2-microglobulin (β2m) light chain, and a peptide of 8-10 amino acid residues (Smith et al., “Model for the In Vivo Assembly of Nascent L^{d} Class I Molecules and for the Expression of Unfolded L^{d} Molecules at the Cell Surface,” J Exp Med 178:2035-2046 (1993); Falk et al., “Allele-Specific Motifs Revealed by Sequencing of Self-Peptides Eluted From MHC Molecules,” Nature 351:290-296 (1991); Schumacher et al., “Direct Binding of Peptide to Empty MHC Class I Molecules on Intact Cells and In Vitro,” Cell 62:563-567 (1990); Townsend et al., “Association of Class I Major Histocompatibility Heavy and Light Chains Induced by Viral Peptides,” Nature 340:443-448 (1989); Van Bleek et al., “Isolation of an Endogenously Processed Immunodominant Viral Peptide From the Class I H-2 Kb Molecule,” Nature 348:213-216 (1990)). Presentation of antigenic peptides by MHC class I at the cell surface requires processing that involves three main steps. The first step is the proteasome-mediated generation of foreign peptides from foreign proteins present in the cytosol. In the second step, the foreign peptides are transported into the endoplasmic reticulum (ER) by proteins known as Transporters Associated with Antigen Processing-1-and 2- (TAP-1 and TAP-2). The two TAP proteins form a heterodimer, and mutations in either TAP gene can prevent antigen presentation by MHC class I molecules. The TAP1 and TAP2 genes map within the MHC itself, and are inducible by interferons, which are produced in response to virus infections. The TAP complex prefers peptides of eight or more amino acids with hydrophobic or basic residues at the carboxy terminus, which corresponds to the type of peptides that bind MHC class I molecules. In the third step of antigen presentation, the foreign peptide binds to the heavy chain/β2m heterodimer of the MHC class I molecule (Pamer et al., “Mechanisms of MHC Class I—Restricted Antigen Processing,” Annu Rev Immunol 16:323-358 (1998); Vukmanovic et al., “Peptide Loading of Nascent MHC Class I Molecules,” Arch Immunol Therap Exp 49:195-201 (2001)).

In the absence of foreign antigens, MHC molecules are occupied by peptides derived from self-proteins. In fact, self-peptides are the major constituent of a pool of MHC-associated peptides even when antigens are presented. Self- or antigenic peptides are held in the groove formed by the β-pleated sheets (floor) and two α-helices (sides) formed by the α1 and α2 domains of the class I heavy chain, as shown in FIG. 1A. The TCR interacts with the top surface of this structure composed of the peptide and both α helices, and in doing so, interacts with residues of both peptide and MHC heavy chain (Bankovich et al., “Not Just Any T Cell Receptor Will Do,” Immunity 18:7-11 (2003); Housset et al., “What Do TCR-pMHC Crystal Structures Teach Us About MHC Restriction and Alloreactivity?” Trends Immunol 24:429-437 (2003); Rudolph et al., “The Specificity of TCR/pMHC Interaction,” Curr Opin Immunol 14:52-65 (2002)).

The TCR is composed of two polypeptide chains, α and β, both of which make contacts with peptide, and, thereby, jointly contribute to the antigen-specificity of the TCR. All peptide/MHC-TCR interactions resolved structurally to date follow the common docking mode differing only by up to 35 degrees in orientation (Bankovich et al., “Not Just Any T Cell Receptor Will Do,” Immunity 18:7-11 (2003); Housset et al., “What Do TCR-pMHC Crystal Structures Teach Us About MHC Restriction and Alloreactivity?” Trends Immunol 24:429-437 (2003); Rudolph et al., “The Specificity of TCR/pMHC Interaction,” Curr Opin Immunol 14:52-65 (2002)). TCR β chain interacts with N-terminal, whereas TCR α chain interacts with C-terminal peptide portion, as shown in FIG. 1B. Variability in the TCR repertoire required for the antigen specificity of the immune responses is generated by combinatorial and junctional diversity generated during rearrangement of TCRα and TCRβ loci (Davis et al., “T-Cell Antigen Receptor Genes and T-Cell Recognition,” Nature 334:395-402 (1988)). Combinatorial diversity is a result of a random joining of distinct gene elements within the TCRα and TCRβ loci, as shown in FIG. 2A. There are twenty-three variable (V), two diversity (D), and twelve joining (J) gene elements in the mouse TCR β locus, and seventy-five Vα and fifty Jα gene elements in the mouse TCRa locus (Arden et al., “Mouse T-cell Receptor Variable Gene Segment Families,” Immunogenetics 42:501-530 (1995)). In the human genome, there are fifty-two V, two D, and thirteen J segments in TCRβ, and seventy V and sixty-one J elements in TCRα locus (Arden et al., “Human T-cell Receptor Variable Gene Segment Families” Immunogenetics 42:455-500 (1995)). Random joining of any V (D; TCR β chain only) to J element, the ability of reading the D segment in all three frames, and random pairing of TCRα and TCRβ chains generates potential combinatorial TCR diversity of 6.21×10⁶ in the mouse and 17.32×10⁶ in a human. This combinatorial diversity is further extended by so-called junctional diversity based on imprecise joining of DNA elements. Thus, template-dependent, as well as template independent (terminal deoxyribonucleotide transferase-mediated), base insertions and/or deletions may occur when these gene elements are joined (Lieber “Site-Specific Recombination in the Immune System,” FASEB J. 5:2934-2944 (1991)). Together, combinatorial and junctional diversity are estimated to contribute to between 1×10¹⁶-1×10¹⁸ distinct TCRs and this constitutes a germline TCR repertoire, as demonstrated in FIG. 2B. The actual TCR repertoire in an individual is a product of selection processes described in more detail below.

The most variable region of the TCR and, therefore, most unique region that determines the specificity of TCR interaction with the ligands is a region called CDR3 (complementarity determining region 3), which is formed by the most C-terminal residues of V region, (D, TCR β chain only) and J regions (Davis et al., “T-Cell Antigen Receptor Genes and T-Cell Recognition,” Nature 334:395-402 (1988)). Because of the variability in the joining of the V(D)J elements (possible additions and deletions) the length of the CDR3 region can be different in individual TCRs. In addition, because of slight differences in the length of individual V segments (Arden et al., “Mouse T-cell Receptor Variable Gene Segment Families,” Immunogenetics 42:501-530 (1995)), the starting position of the CDR3 region is not identical in all TCRs. Generally, CDR3 sequence begins shortly after amino acid 100 and can be about 5-20 amino acids long.

One of the key factors that determines the efficiency of immune response is the affinity/avidity of the TCR for antigen. It is clear that the affinity of binding, but even more importantly, the stability of TCR-peptide/MHC interaction, correlates with the functional potency of T cells (Kersh et al., “High- and Low-Potency Ligands With Similar Affinities for the TCR: The Importance of Kinetics in TCR Signaling,” Immunity 9:817-826 (1998)). Therefore, CD8⁺ T cells expressing high-affinity/avidity TCRs for antigen are key for efficient elimination of infectious agents (Derby et al., “High-Avidity CTL Exploit Two Complementary Mechanisms to Provide Better Protection Against Viral Infection Than Low-Avidity CTL,” J Immunol 166:1690-1697 (2001)) and tumors (Zeh et al., “High Avidity CTLs for Two Self Antigens Demonstrate Superior In vitro and In vivo Antitumor Efficacy,” J Immunol 162:989-994 (1999)). Consequently, generation of high-affinity TCRs has been a goal of immune intervention in these diseases (Dutoit et al., “Functional Analysis of HLA*0201/Melan-S Peptide Multimer+CD8⁺ T Cells Isolated From an HLA-A*0201- Donor: Exploring Tumor Antigen Allorestricted Recognition,” Cancer Immunol 2:7 (2002); Villacres et al., “Relevance of Peptide Avidity to the T Cell Receptor for Cytomegalovirus-Specific Ex Vivo CD8 T Cell Cytotoxicity,” J Infect Dis 188:908-918 (2003)). Measuring the actual affinity of TCR for peptide MHC complexes is not a simple endeavor because both TCR and peptide/MHC are membrane bound proteins. Therefore, at least one component, and preferably both, need to be generated in a soluble form. Because of the importance of the affinity of peptide/MHC-TCR interaction for the outcome of immune responses and the technical difficulty in measuring the actual affinity, the relative affinity/avidity of TCR in many studies is inferred from the concentrations of antigenic peptide required for stimulation of T cells (i.e., the lower the peptide concentration, the higher presumed TCR affinity), or from the functional efficiency of T cells. Although a general correlation between TCR affinity/avidity for peptide/MHC and functional activity of T cells exists, numerous exceptions to the rule have been noted (Gascoigne et al., “T-Cell Receptor Binding Kinetics and T-Cell Development and Activation,” Expert Rev Mol Med 12 February (2001)), suggesting that the two qualities are not necessarily the same. Hence, the term affinity/avidity will be used herein only when actual measurements using soluble peptide/MHC and/or TCR were determined. In other cases, the terms TCR reactivity or TCR sensitivity will be used.

The range of possible affinities of TCR for peptide/MHC complexes is determined during maturation of T cells and TCR repertoire selection in the thymus. MHC molecules are highly polymorphic and TCRs from one individual cannot interact with peptide/MHC molecules from another individual, unless they share the MHC allele(s). This phenomenon is called MHC restriction of the immune responses. The germline TCR repertoire generated as described above by random rearrangements at TCRα and TCRβ loci (Kim et al., “V(D)J Recombination: Site-Specific Cleavage and Repair,” Mol Cell 10:367-374 (2000); Sadofsky, M., “The RAG Proteins in V(D)J Recombination: More Than Just a Nuclease,” Nucleic Acids Res 29:1399-1409 (2001)), is estimated to generate an overwhelming potential TCR diversity of ˜10¹⁶ (Davis et al., “T-Cell Antigen Receptor Genes and T-Cell Recognition,” Nature 334:395-402 (1988)). However, only a minor portion (3-5%) of these TCRs is allowed to be represented in the peripheral lymphoid organs, as shown in FIG. 2B. TCR selection is based on the reactivity of TCRs for self-peptide/MHC complexes expressed in the thymus (Bevan, M., “In Thymic Selection, Peptide Diversity Gives and Takes Away,” Immunity 7:175-178 (1997)). TCRs with relatively high reactivity for the allelic forms of MHC expressed by the host, as well as those with sub-threshold reactivity, are eliminated. The remaining portion of TCRs with intermediate reactivity for self-peptide MHC complexes is selected to form the mature TCR repertoire. Interactions of immature CD4⁺CD8⁺ thymocytes with self-peptide MHC class I complexes will, in general, promote maturation of these cells into CD4-CD8⁺ (simplified CD8⁺) T cells (Koller et al., “Normal Development of Mice Deficient in β2M, MHC Class I Proteins, and CD8⁺ T Cells,” Science 248:1227-1230 (1990); Zijlstra et al., “β2-Microglobulin Deficient Mice Lack CD4-CD8⁺ Cytolytic T Cells,” Nature 344:742-746 (1990)). Conversely, interactions with MHC class II complexes will generally lead to differentiation into CD4⁺CD8- (or simplified CD4⁺) cells (Cosgrove et al., “Mice Lacking MHC Class II Molecule,” Cell 66:1051-1066 (1992); Grusby et al., “Depletion of CD4⁺ T Cells in Major Histocompatibility Complex Class II-Deficient Mice,” Science 253:1417-1420 (1991)). From the pool of the TCRs with intermediate reactivity for self-peptide/MHC, some TCRs will interact with increased affinity with the individual antigen/MHC complexes, and these will be the T cells that will specifically respond to antigen, as shown in FIG. 2C.

There are two types of potential strategies of immunotherapeutic intervention involving TCRs with high reactivity to antigens. The antigen immunization protocol can be theoretically tailored for inducing the high-affinity TCRs. This approach is a golden goal of any vaccination strategy. However, knowledge of the events that lead to successful vaccination is not yet at the level where the affinities of specific TCRs responding to antigen can be accurately predicted. In addition, both tumors and viruses use evasion strategies including, but not limited to, generation of antigen-loss variants (Dudley et al., “Loss of a Unique Tumor Antigen by Cytotoxic T Lymphocyte Immunoselection From a 3-Methylchoantrene-Induced Mouse Sarcoma Reveals Secondary Unique and Shared Antigens,” J Exp Med 184:441-447 (1996); Moore et al., “Evidence of HIV-1 Adaptation to HLA-Restricted Immune Responses at a Population Level,” Science 296:1439-1443 (2002); Pewe et al., “Cytotoxic T Cell-Resistant Variants are Selected in a Virus-Induced Demyelinating Disease,” Immunity 5:253-262 (1996); Pircher et al., “Viral Escape by Selection of Cytotoxic T Cell-Resistant Virus Variants in vivo,” Nature 346:629-633(1990)). This means that even a robust and long-lasting immune response to one antigen determinant does not necessarily mean successful immunity to the antigen as a whole. This is most painfully illustrated in the case of immunity to HIV, where long-term outcome is directly proportional to the number of HIV epitopes to which an immune response is directed (Nelson et al., “Frequency of HLA Allele-Specific Peptide Motifs in HIV-1 Proteins Correlates With the Allele's Association With Relative Rates of Disease Progression After HIV-1 Infection,” Proc Natl Acad Sci USA 94:9802-9807 (1997)). Although most dramatic, HIV infection is not the only example of this correlation. Microbial escape mutants are preferentially selected in the presence of monospecific T cell responses with limited TCR diversity (Franco et al., “Viral Mutations, TCR Antagonism and Escape from the Immune Response,” Curr Opin Immunol 7:524-531 (1995)), and TCR diversity is essential for resistance to viral infection (Messaoudi et al., “Direct Link Between MHC Polymorphism, T Cell Avidity, and Diversity in Immune Defense,” Science 298:1797-1800 (2002)).

To bypass some of the uncertainties associated with vaccination, protocols have been developed that involve isolating successful TCRs in vitro and applying them to patients. This can be achieved either by expanding the self (Dudley et al., “Adoptive-Cell-Transfer Therapy for the Treatment of Patients With Cancer,” Nat Rev Cancer 3:666-675 (2003); Riddell et al., “T-Cell Therapy of CytomegaloVirus and Human Immunodeficiency Virus Infection,” J Antimicrob Chemother 45 (Suppl T3):35-43 (2000)) or allogeneic (Morris et al., “Prospects for Immunotherapy of Malignant Disease,” Clin Exp Immunol 131:1-7 (2003); Stauss, H., “Immunotherapy With CTLs Restricted by Nonself MHC,” Immunol Today 20:180-183 (1999)) T cell clones and injecting them into patients, or by isolating the TCR genes and using them to transduce patients' cells before injection (Willemsen et al., “Genetic Engineering of T Cell Specificity for Immunotherapy of Cancer,” Hum Immunol 64:56-68 (2003)). A disadvantage of both clonal- and gene-based approaches is the limited use of any particular TCR. TCRs that will be used for immunotherapy of melanomas, for example, cannot be used for immunotherapy of any other tumors. Furthermore, because of the differential expression of tumor antigens in the same types of tumors, a particular TCR may not even be suitable for all tumors of the same type. Heterogeneity at the MHC locus in the human population complicates the matter further, as even the same types of tumors with identical rejection tumor antigens but different MHC alleles cannot be treated with the same TCR. Finally, genetic instability and selection of antigen-loss variants of infectious agents and tumors (Dudley et al., “Loss of a Unique Tumor Antigen by Cytotoxic T Lymphocyte Immunoselection From a 3-Methylchoantrene-Induced Mouse Sarcoma Reveals Secondary Unique and Shared Antigens,” J Exp Med 184:441-447 (1996); Moore et al., “Evidence of HIV-1 Adaptation to HLA-Restricted Immune Responses at a Population Level,” Science 296:1439-1443 (2002); Pewe et al., “Cytotoxic T Cell-Resistant Variants are Selected in a Virus-Induced Demyelinating Disease,” Immunity 5:253-262 (1996); Pircher et al., “Viral Escape by Selection of Cytotoxic T Cell-Resistant Virus Variants in vivo,” Nature 346:629-633(1990)) limit the length of beneficial use of a single TCR in a given individual. These circumstances make this type of immunotherapy impractical for wide spread use.

During thymic selection, interaction between TCR and ligands with relatively high sensitivity can result in generation of regulatory cells, rather than in physical elimination (Apostolou et al., “Origin of Regulatory T Cells with Known Specificity for Antigen,” Nat Immunol 3:756-763 (2002); Jordan et al., “Thymic Selection of CD4⁺CD25⁺ Regulatory T Cells Induced by an Agonist Self-Peptide,” Nature Immunol 2:301-306 (2001)). Regulatory cells are predominantly recruited from CD4⁺ T cells and are contained within a subset characterized by cell surface expression of CD25 molecule (Gavin et al., “Control of Immune Homeostasis by Naturally Arising Regulatory CD4⁺ T Cells,” Curr Opin Immunol 15:690-696 (2003)). CD4⁺CD25⁺ T cells are potent inhibitors of in vitro, as well as in vivo, immune responses, and are, consequently, used as a potential source of regulating autoimmune diseases (von Herrath et al., “Antigen-Induced Regulatory T Cells in Autoimmunity,” Nat Rev Immunol 3:223-232 (2003)), allergic reactions and asthma (Akbari et al., “Role of Regulatory T Cells in Allergy and Asthma,” Curr Opin Immunol 15:627-633 (2003)), and graft rejection in transplantation (Wood et al., “Regulatory T Cells in Transplantation Tolerance,” Nat Rev Immunol 3:199-210 (2003)). In addition to CD4⁺CD25⁺T cells, other regulatory cell types have been described that inhibit immune responses via secreting IL-10 and/or TGF-β (Bluestone et al., “Natural Versus Adaptive Regulatory T Cells,” Nat Rev Immunol 3:253-257 (2003)). The manner in which these other types of regulatory cells are selected is presently unknown. Thus, one might anticipate that CD4⁺ T cells with high-sensitivity TCR repertoire will contain enhanced regulatory activity.

The contemporary adoptive T cell immunotherapy of tumors and infectious diseases is based on in vitro identification, selection, and expansion of T cells with TCRs of presumed high affinity/avidity for antigen, followed by injection of expanded cells into patients (Dudley et al., “Adoptive-Cell-Transfer Therapy for the Treatment of Patients With Cancer,” Nat Rev Cancer 3:666-675 (2003)). Because TCRs with high affinity/avidity are infrequently encountered within populations of antigen-specific cells (and this is especially true in the case of tumor antigens, which are, most of the time, self-antigens), presence of high affinity/avidity TCR in injected cells has been sought via transducing peripheral blood cells with TCRs of known specificity and avidity (Willemsen et al., “Genetic Engineering of T Cell Specificity for Immunotherapy of Cancer,” Hum Immunol 64:56-68 (2003)), or by using allogeneic T cell donors of peptide-specific allo-reactive T cells (Morris et al., “Prospects for Immunotherapy of Malignant Disease,” Clin Exp Immunol 131:1-7 (2003)). The drawback of both of these strategies is in the limited use of one TCR (whether transfected or selected in vitro) for one antigen and presenting MHC molecule. Diversity of possible antigens and MHC alleles requires great numbers of distinct TCRs.

To date no way has been shown to harness the potential advantage of TCRs with high reactivity to antigens with passive transfer of TCRs (either as genes or T cell clones) and bypass the need for isolating many different specific TCRs. What is needed is a method to derive high affinity TCRs without directing antigen specificity and MHC restriction. Such a method would be highly useful for treatment of infectious and proliferative diseases and would also provide a method directed to the treatment of autoimmune disease by enhancing the population of regulatory T cells in a subject.

The present invention is directed to overcoming these and other deficiencies in the art.

### SUMMARY OF THE INVENTION

The present invention relates to a method of treating a disease caused by infectious agents or disregulated proliferation of a cell type in a subject. This method involves providing a T cell receptor β chain having higher sensitivity recognition of antigen than a wild type T cell receptor β chain and introducing the T cell receptor β chain having higher sensitivity recognition of antigen to a subject having the disease under conditions effective to treat the disease.

The present invention also relates to a method of treating a lymphocyte-mediated disease in a subject. This method involves providing a T cell receptor β chain having higher sensitivity recognition of antigen than does a wild type T cell receptor β chain and introducing the T cell receptor β chain having higher sensitivity recognition of antigen to a subject having the lymphocyte-mediated disease under conditions effective to treat the lymphocyte-mediated disease.

The present invention also relates to a transgenic mouse having a T cell receptor β chain having higher sensitivity recognition of antigen than a wild type mouse.

The present invention also relates to another method of treating a disease caused by infectious agents or disregulated proliferation of a cell type in a subject. This method involves providing an isolated mouse T cell receptor β chain having higher sensitivity recognition of antigen than a wild type T cell receptor β chain, linking the mouse T cell receptor β chain having higher sensitivity recognition of antigen with a human T cell receptor a chain, and introducing the linked mouse T cell receptor β chain and human T cell receptor a chain to a subject having the disease under conditions effective to treat the disease.

The present invention also relates to another method of treating a lymphocyte-mediated disease in a subject. This method involves providing an isolated mouse T cell receptor β chain having higher sensitivity recognition of antigen than a wild type T cell receptor β chain, linking the mouse T cell receptor β chain having higher sensitivity recognition of antigen with a human T cell receptor α chain, and introducing the linked mouse T cell receptor β chain and human T cell receptor a chain to a subject having the disease under conditions effective to treat the disease.

The present invention provides a method of producing a high sensitivity TCR repertoire characterized by great flexibility and a wide potential for application that circumvents the shortcomings of other strategies, without directing antigen specificity and MHC restriction. This approach is superior to those currently in existence by virtue of using one component of the TCR, rather than the whole TCR, that imparts greater sensitivity of ligand recognition. The TCR β chain of the present invention can be used on its own, or in combination with many existing approaches. In addition, when used in a manner to allow thymic differentiation, the TCR β chain is expected to impart the greater reactivity of regulatory T cells, which can be used for dampening acute or chronic lymphocyte mediated diseases.

### BRIEF DESCRIPTION OF THE DRAWINGS

FIGS. 1A-B are diagrams of the structure of the TCR interacting with peptide/MHC complex. FIG. 1A shows the TCR (top portion) binds to the top surface of peptide (indicated by tube extending from P1-P8) held in the MHC class I groove formed by the two a helices positioned on the edges of the surface formed by antiparallel β pleated sheets. The loops of the TCR contacting the ligand (CDR loops) are designated by arrows. FIG. 1B shows the TCR antigen-binding site superimposed upon the top surface of the peptide/MHC complex. The areas contacted by distinct CDR loops are indicated.

FIGS. 2A-C show the generation of antigen-specific TCR repertoire. FIG. 2A illustrates the process of random rearrangement of one of the many variable (V), diversity (D), and junctional (J) elements present in the TCRβ locus and creation of unique sequence responsible for specificity of interaction with the TCR ligands. A completely identical process occurs at the TCRα locus, except that that there are no diversity elements. The diagram shown in FIG. 2B demonstrates the concept that through random rearrangement of one of the many variable, diversity, and junctional elements present in the TCRβ and TCRα loci (combinatorial diversity), and their imprecise joining (junctional diversity), a germline repertoire of ˜1×10¹⁶ different TCRs is generated. Only about 20% of these TCRs have at least an intermediate affinity for self-peptide/MHC complexes present in the thymus of an individual, and these TCRs will be selected for maturation. However, the affinity of a large portion of these TCRs for peptide/MHC will be too high, so that a large portion of these TCRs will be deleted to avoid potential autoimmune reactions. In this manner, ˜3-5% of the primary germline TCR repertoire is ultimately selected to form the peripheral TCR repertoire. FIG. 2C shows that a TCR selected in the thymus based on an intermediate affinity for self-peptide/MHC complex will be activated in the periphery if a foreign antigen has structural features allowing high affinity interaction.

FIG. 3 is a schematic representation of structural features of four distinct TCRs, and shows the selection of TCR with high sensitivity recognition in a β2m-deficient (“β2m −/−”) environment. Arrows point to the subtle differences in the ligand-binding region of the TCRs 2-4. These structural features enable tighter interactions with complementary structures of some peptides (TCRs 2-3), or of MHC molecule itself (TCR 4).

FIG. 4 is a schematic representation of interactions of distinct TCRs with endogenous and exogenous ligands, and the functional consequences based on the extent of established contacts. TCR 1-3 represent “peptide-specific” TCRs that require strong interaction with peptides. TCR4 represents the “MHC-based” TCR of the present invention that receives much of the energy for binding from the MHC molecule itself. Normally, because of the high peptide diversity (on average 1×10 ³ different species per cell) and limited MHC diversity (an individual human may have up to six different MHC class I and class II molecules), “peptide-specific” TCRs are much more abundant. MHC-based TCRs show higher sensitivity of antigen recognition, because they can tolerate an imperfect fit with the peptide portion of the ligand. Compensation by tighter interaction with the MHC molecule also enables “promiscuous” selection of MHC-based TCRs.

FIGS. 5A-B show how MHC class I density of wild type and β2m-deficient thymic epithelial cells, respectively, determine predominant selection of peptide-specific or MHC-based TCRs.

FIGS. 6A-D are graphs showing the reactivity of H-2K^{d} allospecific CD8⁺ T cells from MHC-deficient or wild type mice to TAP-deficient targets. FIGS. 6A-B show the allele-specificity of allo-reactive CD8+ T cell lines obtained from β2m−/− (line MD5) or wild type (line B6X) mice, respectively, tested in a chromium release assay using fibroblastoid cell line MC57G, as well as the clones of this cell line transfected H-2K^{d} or H-2L^{d}, as targets. FIGS. 6C-D show the requirement of functional TAP transporters in the target cells for efficient lysis by the same CD8⁺ cell lines, tested using RMA-S cells and appropriate H-2K^{d} or H-2L^{d} transfected variants.

FIGS. 7A-B show the analysis of the TCR usage in β2m−/− CD8+ T cell lines. FIG. 7A is a photo of a gel showing RT-PCR analysis of TCR a (lanes 1-3 and 6-8; lanes 1,6-TCR α primer set A; lanes 2,7-TCR α primer set B; lanes 3,8-TCR-a primer set C), TCR β (lanes 4 and 9) and β-actin (control) (lanes 5 and 10) expression in line MD5 cells, or in unseparated wild type thymocytes. Products were visualized by adding 35^{S}-labeled dATP at the beginning of reaction, and resolved using 5% polyacrylamide electrophoresis gel (PAGE). RT-PCR products from wild type thymus, MD5 cells (β2m −/−), or CD8⁺ cell lines (see Table 2, below) obtained from β2m −/− mice grafted with the wild type thymic epithelium (TG-thymus grafted) were cloned and sequenced. FIG. 7B shows the diversity of the sequences obtained from CD8⁺ T cell lines from β2m −/− mice or β2m −/− mice grafted with WT thymus in the function of time (weeks of in vitro cultivation is indicated).

FIGS. 8A-C show the generation and primary analysis of T cell subsets in TCR β transgenic mice (MTB mice). FIG. 8A is a schematic representation of the TCR β construct. TCRβ cDNA (arrow) was placed under the control of a mouse H-2 promoter/Ig enhancer-containing DNA segment in the pHSE3′ vector. The human β-globin gene fragment provides the cDNA with an intron and poly-adenylation signal. FIG. 8B shows histograms of the expression of TCRβ transgene in the transgenic mice, as revealed by immunofluorescent staining of peripheral blood leukocytes using the Vβ2-specific monoclonal antibody. Shown are the profiles of a transgenic mouse (lower histogram) and of a nontransgenic littermate (upper histogram). FIG. 8C is the immunofluorescence analysis of T cell subsets in the periphery and the thymus of transgenic mouse (MTB) and of a nontransgenic littermate (WT). Cells were stained using CD4- and CD8-specific monoclonal antibodies and analyzed by flow cytometry. Shown are dot-plots with percent of cells present in indicated quadrants.

FIGS. 9A-C are the characterization of allogeneic responses in MTB mice. Wild type or MTB (two individual) spleen cell populations (H-2 b) were stimulated for five days in vitro with irradiated BALB/c (H-2^{d}) spleen cells. FIG. 9A shows the cytotoxic activity of cultured cells against P815 (H-2^{d}) or EL4 (H-2^{b}) targets at indicated effector to target ratios determined at the end of the culture period. In FIG. 9B, wild type or MTB spleen cells were stimulated for five days in vitro with irradiated BALB/c spleen cells under limiting dilution conditions. At the end of the culture ⁵¹Cr-labeled P815 cells were added to the wells and release of ⁵¹Cr after a four hour incubation was determined. Specific lysis obtained in each individual well is shown. Mean values are indicated by horizontal lines. In FIG. 9C, wild type (B6) or MTB spleen cells were stimulated as in FIG. 9A and then tested for lytic (cytotoxic) activity against ⁵¹Cr-labeled MC57G (H-2^{b}) targets or clones transfected with either H-2K^{d} or H-2L^{d}.

FIGS. 10A-D are cytotoxic T-lymphocyte (CTL) assay results showing that MTB cells display enhanced recognition of alloantigens presented by TAP-deficient cells. Wild type or MTB spleen cells (H-2^{b}) were stimulated for five days in vitro with irradiated BALB/c (H-2^{d}) spleen cells. FIG. 10A shows the cytotoxic activity of cultured cells against ⁵¹Cr-labeled RMA-S(H-2^{b}), RMA-S-K^{d} or RMA-S-L^{d} determined in a CTL assay. Wild type or MTB spleen cells were stimulated for five days in vitro with irradiated RMA-S-L^{d} cells. FIG. 10B shows the cytolytic activity against the same cells, or parental RMA-S cells. FIGS. 10C-D demonstrate that alloantigens displayed by P815 and RMA-S-L^{d} cells are not shared. MTB spleen cells were stimulated for five days in vitro with irradiated BALB/c spleen cells (FIG. 10C) or RMA-S-L^{d} cells (FIG. 10D). Cultured cells were then tested for CTL activity against indicated targets. RMA is a TAP-sufficient, parental version of RMA-S cells and is of H-2^{b} haplotype.

FIGS. 11 A-B show the enhanced selection of MTB CD8⁺ T cells on TAP-1-deficient background. MTB mice were bred to TAP1−/− background. Spleen cells and thymocytes of the resulting strain were stained with CD4- and CD8-specific monoclonal antibodies and the profiles obtained were compared to the TAP1−/− controls. Representative dot-plots of flow cytometry analysis are shown in FIG. 11A. Percent cells present in each quadrant is indicated. FIG. 11B is a bar graph showing mean and standard deviations of percent CD8⁺ T cells found in five individual mice of each genotype resulting when MTB mice were bred to TAP1−/− background.

FIG. 12A-C are histograms showing CD5 expression in MTB and WT cells from different sources. MTB and WT thymocytes, spleen, and lymph node cells were stained with anti-CD4, anti-CD8, and anti-CD5 monoclonal antibodies and analyzed by flow cytometry. FIG. 12A shows overlay histograms of CD5 expression in WT (dotted line) and MTB (plain line) thymocytes gated for CD4 and CD8 expression. FIG. 12B shows overlay histograms of CD5 expression in WT (dotted line) and MTB (plain line) lymph node cells gated for CD4 expression (CD4⁺ lymph node cells). FIG. 12C shows overlay histograms of CD5 expression in WT (dotted line) and MTB (plain line) lymph node cells gated for CD8 expression (CD8⁺ lymph node cells).

FIGS. 13A-B demonstrate that the transfer of MTB spleen cells protects WT mice from TAP-deficient tumor growth. C57BL/6 mice were injected with PBS, or indicated numbers of WT or MTB spleen cells i.v., and challenged with 1×10⁶ live RMA-S-L^{d} tumor s.c. Mice were checked three times a week for tumor growth and sacrificed if the tumor diameter exceeded 2 cm, or if mice exhibited cachexia. FIG. 13A shows the percent survival. FIG. 13B shows mean tumor volume with standard error in each experimental group.

FIGS. 14A-B are graphs showing that MTB mice are more susceptible than WT mice to the growth of TAP-deficient tumor. C57BL/6 or MTB mice were injected with PBS, or 2×10⁷ irradiated RMA-S-L^{d} cells i.p. Four weeks later mice were challenged with 1×10⁶ live RMA-S-L^{d} tumor s.c. Mice were checked three times a week for tumor growth and sacrificed if the tumor diameter exceeded 2 cm, or mice exhibited cachexia. FIG. 14A shows the percent survival WT versus MTB mice in each experimental group. FIG. 14B shows mean tumor volume in each experimental group with standard error.

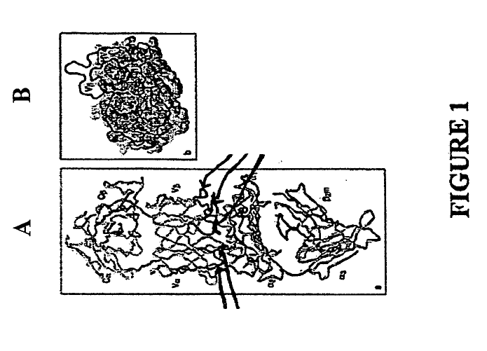

### DETAILED DESCRIPTION OF THE INVENTION

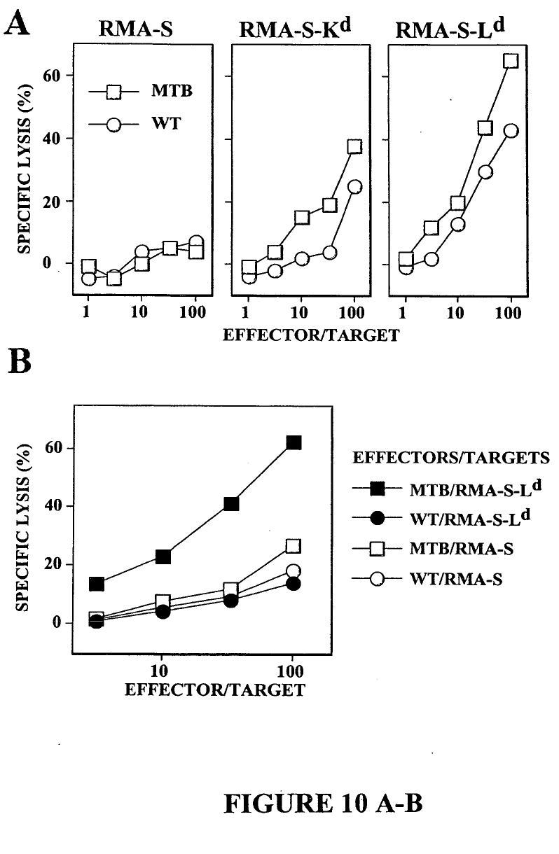

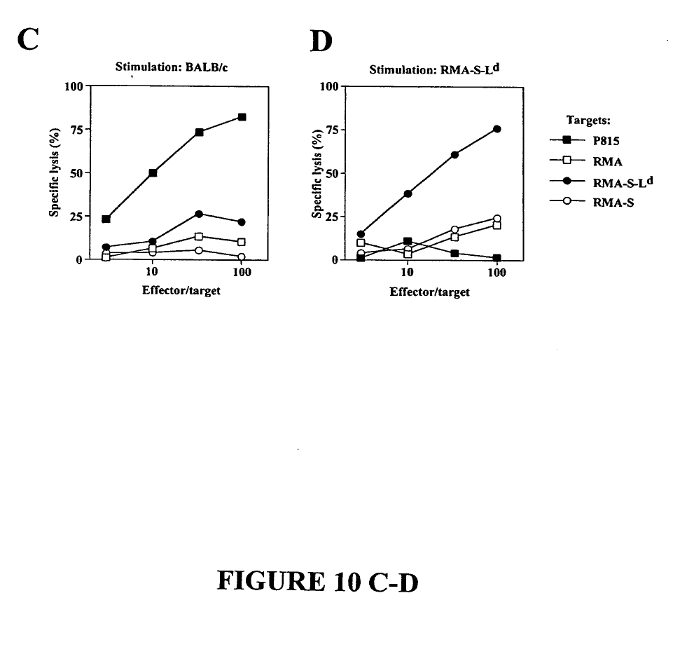

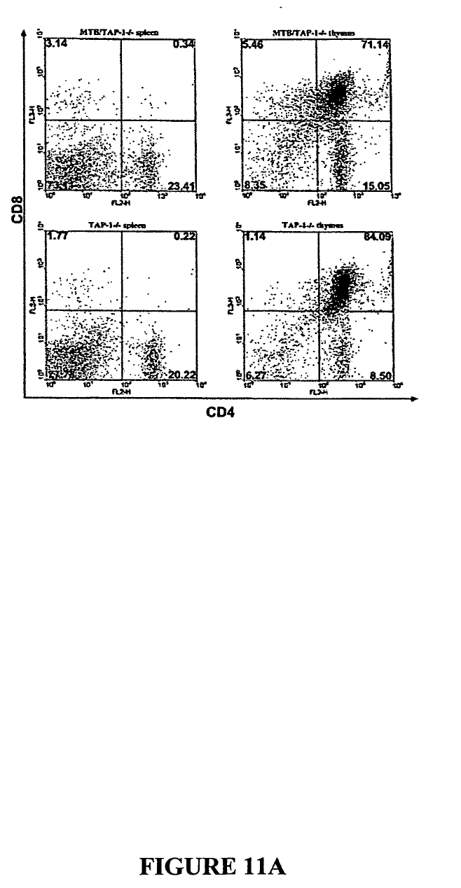

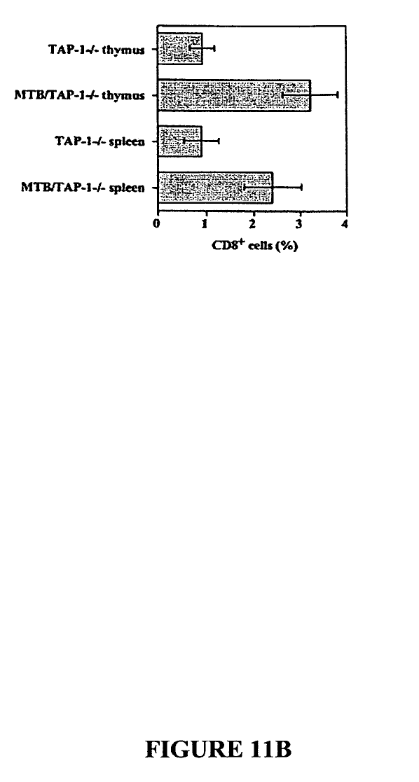

One aspect of the present invention relates to a method of treating a disease caused by infectious agents or disregulated proliferation of a cell type in a subject. This method involves providing a T cell receptor β chain having higher sensitivity recognition of antigen than a wild type T cell receptor β chain and introducing the T cell receptor β chain having higher sensitivity recognition of antigen to a subject having a disease under conditions to treat the disease. A “higher sensitivity” T cell receptor β chain or TCR repertoire as used herein is defined by higher functional potency when compared to the wild type. This higher functional potency (i.e., higher sensitivity interactions with ligand), is illustrated by the ability to respond to antigens presented by cells with low levels of MHC class I (as shown in FIG. 10B) and by superior maturation of T cells in the thymus expressing low levels of MHC class I (seen in FIGS. 11A-B). The superior functional ability is most likely a consequence of an increase in affinity/avidity of the TCR for MHC I over the average affinity/avidity of TCR repertoire in a wild type mouse.

One form of the T cell receptor β chain of present invention having higher sensitivity recognition of antigen than a wild type T cell receptor β chain is encoded by a nucleic acid molecule having a nucleotide sequence corresponding to SEQ ID NO: 1, as follows:

| | |
|---|---|
| atgtggcagt tttgcattct gtgcctctgt gtactcatgg cttctgtggc tacagacccc | 60 |
| acagtgactt tgctggagca aaacccaagg tggcgtctgg taccacgtgg tcaagctgtg | 120 |
| aacctacgct gcatcttgaa gaattcccag tatccctgga tgagctggta tcagcaggat | 180 |
| ctccaaaagc aactacagtg gctgttcact ctgcggagtc ctggggacaa agaggtcaaa | 240 |
| tctcttcccg gtgctgatta cctggccaca cgggtcactg atacggagct gaggctgcaa | 300 |
| gtggccaaca tgagccaggg cagaaccttg tactgcacct gcagtgcata ctggggtgga | 360 |
| aacaccttgt actttggtgc gggcacccga ctatcggtgc tagaggatct gagaaatgtg | 420 |
| actccaccca aggtctcctt gtttgagcca tcaaaagcag agattgcaaa caaacaaaag | 480 |
| gctaccctcg tgtgcttggc caggggcttc ttccctgacc acgtggagct gagctggtgg | 540 |
| gtgaatggca aggaggtcca cagtggggtc agcacggacc ctcaggccta caaggagagc | 600 |
| aattatagct actgcctgag cagccgcctg agggtctctg ctaccttctg gcacaatcct | 660 |
| cgaaaccact tccgctgcca agtgcagttc catgggcttt cagaggagga caagtggcca | 720 |
| gagggctcac ccaaacctgt cacacagaac atcagtgcag aggcctgggg ccgagcagac | 780 |
| tgtggaatca cttcagcatc ctatcatcag ggggttctgt ctgcaaccat cctctatgag | 840 |
| atcctactgg ggaaggccac cctatatgct gtgctggtca gtggcctggt gctgatggcc | 900 |
| atggtcaaga aaaaaaattc ctga | 924 |

The nucleic acid molecule having SEQ ID NO: 1 encodes a protein or polypeptide having an amino acid sequence corresponding to SEQ ID NO: 2, as follows:

```
Met Trp Gln Phe Cys Ile Leu Cys Leu Cys Val Leu Met Ala Ser Val
    1 5 10 15
Ala Thr Asp Pro Thr Val Thr Leu Leu Glu Gln Asn Pro Arg Trp Arg
                          20 25 30
Leu Val Pro Arg Gly Gln Ala Val Asn Leu Arg Cys Ile Leu Lys Asn
                  35 40 45
Ser Gln Tyr Pro Trp Met Ser Trp Tyr Gln Gln Asp Leu Gln Lys Gln
          50 55 60
Leu Gln Trp Leu Phe Thr Leu Arg Ser Pro Gly Asp Lys Glu Val Lys
  65 70 75 80
Ser Leu Pro Gly Ala Asp Tyr Leu Ala Thr Arg Val Thr Asp Thr Glu
                                  85 90 95
Leu Arg Leu Gln Val Ala Asn Met Ser Gln Gly Arg Thr Leu Tyr Cys
                        100 105 110
Thr Cys Ser Ala Tyr Trp Gly Gly Asn Thr Leu Tyr Phe Gly Ala Gly
                115 120 125
Thr Arg Leu Ser Val Leu Glu Asp Leu Arg Asn Val Thr Pro Pro Lys
        130 135 140
Val Ser Leu Phe Glu Pro Ser Lys Ala Glu Ile Ala Asn Lys Gln Lys
145 150 155 160
Ala Thr Leu Val Cys Leu Ala Arg Gly Phe Phe Pro Asp His Val Glu
                                165 170 175
Leu Ser Trp Trp Val Asn Gly Lys Glu Val His Ser Gly Val Ser Thr
                        180 185 190
Asp Pro Gln Ala Tyr Lys Glu Ser Asn Tyr Ser Tyr Cys Leu Ser Ser
                195 200 205
Arg Leu Arg Val Ser Ala Thr Phe Trp His Asn Pro Arg Asn His Phe
        210 215 220
Arg Cys Gln Val Gln Phe His Gly Leu Ser Glu Glu Asp Lys Trp Pro
225 230 235 240
Glu Gly Ser Pro Lys Pro Val Thr Gln Asn Ile Ser Ala Glu Ala Trp
                                245 250 255
Gly Arg Ala Asp Cys Gly Ile Thr Ser Ala Ser Tyr His Gln Gly Val
                        260 265 270
Leu Ser Ala Thr Ile Leu Tyr Glu Ile Leu Leu Gly Lys Ala Thr Leu
                275 280 285
Tyr Ala Val Leu Val Ser Gly Leu Val Leu Met Ala Met Val Lys Lys
        290 295 300
Lys Asn Ser
305
```

Mutations or variants of the above polypeptides or proteins are encompassed by the present invention. Variants may be modified by, for example, the deletion or addition of amino acids that have minimal influence on the properties, secondary structure, and hydropathic nature of the desired polypeptide. For example, as described herein above, variability in the TCR repertoire required for the antigen specificity of the immune responses is generated by combinatorial and junctional diversity generated during rearrangement of TCR α and TCR β loci (Davis et al., “T-Cell Antigen Receptor Genes and T-Cell Recognition,” Nature 334:395-402 (1988), which is hereby incorporated by reference in its entirety). In particular, variability in the joining of the V(D)J elements (possible additions and deletions), and differences in the length of individual V segments (Arden et al., “Mouse T-cell Receptor Variable Gene Segment Families,” Immunogenetics 42:501-530 (1995), which is hereby incorporated by reference in its entirety), can result in CDR3 regions that are different in individual TCRs, including TCRs in which the starting position of the CDR3 region varies, or in which the length of the CDR3 region varies among TCRs. These variations, however, can occur without varying the specificity of TCR interaction, i.e., the sensitivity of the TCR. In the present invention, the polypeptide or protein of the present invention may vary considerably, provided the WGG motif located in the CDR3 region (at positions 118-120 in SEQ ID NO: 2) is present in the CDR3 region of the TCR β chain.

In addition, the polypeptide or protein of the present invention may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Fragments of the above proteins are also encompassed by the present invention. Suitable fragments can be produced by several means. In the first, subclones of the gene encoding the desired protein of the present invention are produced by conventional molecular genetic manipulation by subcloning gene fragments. The subclones then are expressed in vitro or in vivo in a suitable host, for example, bacterial cells, to yield a smaller protein or peptide.

In another approach, based on knowledge of the primary structure of the proteins of the present invention, fragments of the genes of the present invention may be synthesized by using the polymerase chain reaction (“PCR”) technique together with specific sets of primers chosen to represent particular portions of the protein. These then would be cloned into an appropriate vector for increased expression of an accessory peptide or protein.

Chemical synthesis can also be used to make suitable fragments. Such a synthesis is carried out using known amino acid sequences for the proteins of the present invention. These fragments can then be separated by conventional procedures (e.g., chromatography, SDS-PAGE) and used to practice the present invention.

The nucleic acid molecule encoding the TCR β chain of the present invention may be introduced to a subject directly, entering appropriate host cells in vivo (using gene therapy, also referred to herein as the “direct” method of gene introduction), or indirectly, by introducing it into appropriate recipient cells in vitro followed by injection of transgenic cells into a subject. In this and all aspects of the present invention, a “subject” includes any mammal, without limitation, including a human.

Thus, in one aspect of the present invention, the nucleic acid molecule encoding the TCR β chain of the present invention is first inserted into a cell and the transgenic cell is introduced into the subject. The nucleic acid molecule encoding a high sensitivity TCR β chain polypeptide or protein of the present invention can be introduced into an expression system of choice using conventional recombinant technology. Generally, this involves inserting the nucleic acid molecule into an expression system to which the molecule is heterologous (i.e., not normally present). The introduction of a particular foreign or native gene into a mammalian host is facilitated by first introducing the gene sequence into a suitable nucleic acid vector. “Vector” is used herein to mean any genetic element, such as a plasmid, phage, transposon, cosmid, chromosome, virus, virion, etc., which is capable of replication when associated with the proper control elements and which is capable of transferring gene sequences between cells. Thus, the term includes cloning and expression vectors, as well as viral vectors. The heterologous nucleic acid molecule is inserted into the expression system or vector in proper sense (5′→3′) orientation and correct reading frame. Alternatively, the nucleic acid may be inserted in the “antisense” orientation, i.e, in a 3′→5′ prime direction. The vector contains the necessary elements for the transcription and translation of the inserted protein-coding sequences.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Recombinant genes may also be introduced into viruses, including vaccinia virus, adenovirus, and retroviruses, including lentivirus. Recombinant viruses can be generated by transfection of plasmids into cells infected with virus.

Suitable vectors include, but are not limited to, the following viral vectors such as lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference in its entirety), pQE, pIH821, pGEX, pET series (F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual, Cold Springs Laboratory, Cold Springs Harbor, N.Y. (1989), which is hereby incorporated by reference in its entirety.

A variety of host-vector systems may be utilized to express the protein-encoding sequence of the present invention. Primarily, the vector system must be compatible with the host cell used. Host-vector systems include, but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (“mRNA”) translation).

Transcription of DNA is dependent upon the presence of a promoter which is a DNA sequence that directs the binding of RNA polymerase and thereby promotes mRNA synthesis. The DNA sequences of eukaryotic promoters differ from those of prokaryotic promoters. Furthermore, eukaryotic promoters and accompanying genetic signals may not be recognized in or may not function in a prokaryotic system, and, further, prokaryotic promoters are not recognized and do not function in eukaryotic cells.

Similarly, translation of mRNA in prokaryotes depends upon the presence of the proper prokaryotic signals which differ from those of eukaryotes. Efficient translation of mRNA in prokaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression see Roberts and Lauer, Methods in Enzymology, 68:473 (1979), which is hereby incorporated by reference in its entirety.

Promoters vary in their “strength” (i.e., their ability to promote transcription). For the purposes of expressing a cloned gene, it is desirable to use strong promoters in order to obtain a high level of transcription and, hence, expression of the gene. Depending upon the host cell system utilized, any one of a number of suitable promoters may be used. For instance, when cloning in E. coli, its bacteriophages, or plasmids, promoters such as the T7 phage promoter, lac promoter, trp promoter, recA promoter, ribosomal RNA promoter, the P_{R} and P_{L} promoters of coliphage lambda and others, including but not limited, to lacUV5, ompF, bla, lpp, and the like, may be used to direct high levels of transcription of adjacent DNA segments. Additionally, a hybrid trp-lacUV5 (tac) promoter or other E. coli promoters produced by recombinant DNA or other synthetic DNA techniques may be used to provide for transcription of the inserted gene.

Bacterial host cell strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in prokaryotic cells. These transcription and translation initiation signals may vary in “strength” as measured by the quantity of gene specific messenger RNA and protein synthesized, respectively. The DNA expression vector, which contains a promoter, may also contain any combination of various “strong” transcription and/or translation initiation signals.

Depending on the vector system and host utilized, any number of suitable transcription and/or translation elements, including constitutive, inducible, and repressible promoters, as well as minimal 5′ promoter elements may be used. Regulatory DNA sequences including, but not limited to promoters that drive CD8⁺ and CD4⁺ T-cell-specific expression are particularly useful in the expression constructs of the present invention (Kieffer et al., “Identification of a Candidate Regulatory Region in the Human CD8 Gene Complex by Colocalization of DNase I Hypersensitive Sites and Matrix Attachment Regions which Bind SATB1 and GATA-3,” J Immunol 168(8):3915-22 (2002), Marodon et al., “Specific Transgene Expression in Human and Mouse CD4⁺ Cells Using Lentiviral Vectors with Regulatory Sequences from the CD4 Gene,” Blood 101(9):3416-23 (2003) which are hereby incorporated by reference in their entirety).

The nucleic acid molecule(s) of the present invention, a regulatory DNA sequence of choice, a suitable 3′ regulatory region, and if desired, a reporter gene, are incorporated into a vector-expression system of choice to prepare the nucleic acid construct of present invention using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

In one aspect of the present invention, a nucleic acid molecule encoding a protein of choice is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of a suitable promoter, to prepare a nucleic acid construct of the present invention. The nucleic acid molecule may be inserted into the expression system or vector in the antisense (i.e., 3′→5′) orientation for use in other aspects.

Once the isolated nucleic acid molecule encoding the TCR protein or polypeptide of the present invention has been cloned into an expression system, it is ready to be incorporated into a host cell. Recombinant molecules can be introduced into cells via transformation, particularly transduction, conjugation, lipofection, protoplast fusion, mobilization, particle bombardment, or electroporation. The DNA sequences are cloned into the host cell using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety. Suitable hosts include, but are not limited to, bacteria, virus, yeast, fungi, mammalian cells, and insect cells, plant cells, and the like. Suitable mammalian cells include peripheral blood leukocytes (PBLs), as a source of peripheral T-cells, and hematopoietic progenitor cells, including, without limitation, bone marrow cells, isolated bone marrow stem cells, umbilical cord blood cells, and granulocyte-colony stimulating factor (G-CSF)-mobilized peripheral blood leukocytes.

Transient expression in protoplasts allows quantitative studies of gene expression since the population of cells is very high (on the order of 10⁶). To deliver DNA inside protoplasts, several methodologies have been proposed, but the most common are electroporation (Neumann et al., “Gene Transfer into Mouse Lyoma Cells by Electroporation in High Electric Fields,” EMBO J. 1: 841-45 (1982); Wong et al., “Electric Field Mediated Gene Transfer,” Biochem Biophys Res Commun 30;107(2):584-7 (1982); Potter et al., “Enhancer-Dependent Expression of Human Kappa Immunoglobulin Genes Introduced into Mouse pre-B Lymphocytes by Electroporation,” Proc. Natl. Acad. Sci. USA 81: 7161-65 (1984), which are hereby incorporated by reference in their entirety) and polyethylene glycol (PEG) mediated DNA uptake, Sambrook et al., Molecular Cloning: A Laboratory Manual, Chap. 16, Second Edition, Cold Springs Laboratory, Cold Springs Harbor, N.Y. (1989), which is hereby incorporated by reference in its entirety). During electroporation, the DNA is introduced into the cell by means of a reversible change in the permeability of the cell membrane due to exposure to an electric field. PEG transformation introduces the DNA by changing the elasticity of the membranes. Unlike electroporation, PEG transformation does not require any special equipment and transformation efficiencies can be equally high. Another appropriate method of introducing the gene construct of the present invention into a host cell is fusion of protoplasts with other entities, either minicells, cells, lysosomes, or other fusible lipid-surfaced bodies that contain the chimeric gene (Fraley et al., “Entrapment of a Bacterial Plasmid in Phospholipid Vesicles: Potential for Gene Transfer,” Proc Natl Acad Sci USA 76(7):3348-52 (1979); Fraley et al., “Introduction of Liposome-Encapsulated SV40 DNA into Cells,” J Biol Chem 255(21):10431-10435 (1980), which are hereby incorporated by reference in the entirety).

Stable transformants are preferable for the methods of the present invention, which can be achieved by using variations of the methods above as describe in Sambrook et al., Molecular Cloning: A Laboratory Manual, Chap. 16, Second Edition, Cold Springs Laboratory, Cold Springs Harbor, N.Y. (1989), which is hereby incorporated by reference in its entirety.

Typically, an antibiotic or other compound useful for selective growth of the transformed cells only is added as a supplement to the media. The compound to be used will be dictated by the selectable marker element present in the plasmid with which the host cell was transformed. Suitable genes are those which confer resistance to gentamycin, G418, hygromycin, streptomycin, spectinomycin, tetracycline, chloramphenicol, and the like. Similarly, “reporter genes,” which encode enzymes providing for production of an identifiable compound, or other markers which indicate relevant information regarding the outcome of gene delivery, are suitable. For example, various luminescent or phosphorescent reporter genes are also appropriate, such that the presence of the heterologous gene may be ascertained visually. The selection marker employed will depend on the target species and/or host or packaging cell lines compatible with a chosen vector.

After the transgenic host cells are identified, they are grown to a desired density in cell culture media appropriate for the cell type, under conditions suitable for the maintenance and, if desired, expansion of the cell population prior to the application of the cells in accordance with the methods of the present invention.

The simplest method of introducing the high affinity TCRβ chain of the present invention to subjects is by in vitro transduction of cells with the TCRβ chain of the present invention as described above, and subsequent injection of the transduced cells into patients. This is referred to as the “indirect” method of gene introduction hereinafter. Different cell types are suitable for in vitro transduction, including, without limitation, peripheral cells and bone marrow derived cells. Therefore, in one aspect of the present invention, the high-sensitivity TCR repertoire of the present invention is introduced indirectly, by injecting the subject with in vitro transduced T cells. In this aspect, peripheral blood leukocytes (PBLs), a source of peripheral T-cells (and, potentially, hematopoietic progenitor cells), are transfected with the nucleic acid molecule encoding the high sensitivity TCRβ chain of the present invention prepared in a suitable expression vector as described above. In this “peripheral strategy,” as it is referred to hereinafter, the regulatory component of thymic selection is bypassed, eliminating TCRs with high affinity/avidity to self MHC/peptide complexes. This strategy will therefore have a special advantage for use in immunotherapy of tumors, where antigens are frequently self-peptides. Thus, this aspect includes, without limitation, treatment of the following cancers: melanoma, breast cancer, prostate cancer, leukemias, lymphomas, mastocytoma, plasmocytoma, multiple myeloma, lung tumor (adenocarcinoma), ovarian cancer, testicular cancer, stomach cancer, intestinal cancer, neuroblastoma, pheochromocytoma, Wilms tumor, renal cell carcinoma, osteosarcoma, Ewing sarcoma, retinoblastoma, medulloblastoma, nasopharyngeal carcinoma, pancreatic carcinoma, hepatoblastoma, hepatoma, and cervical adenocarcinoma.

In addition, the present invention is highly suitable for the treatment of diseases caused by a variety of infectious agents, including, without limitation, AIDS, hepatitis A, B, and C, cytomegalovirus, infectious mononucleosis, influenza, herpes, Varicella-zoster, yellow fever, dengue fever, smallpox, Rous sarcoma virus (RSV), listeriosis, tuberculosis, leprosy, brucellosis, Legionnaire's disease, chlamydial infections, Rickettsial diseases, cholera, anthrax, Lyme disease, malaria, toxoplasmosis, giardiasis, trypanosomiasis, leishmaniasis, shistosomiasis, filariasis, candidiasis and other mycotic infections, cryptosporidium, microsporidium, histoplasma capsulatum, pneumocystis carinii, Cryptococcus neoformans, Coccidioides immitis, and helminic infections. There are a number of infectious diseases which have distinct clinical outcome depending on the MHC class I or class II alleles displayed by the host, although the mechanism(s) of association have not been resolved yet (Vukmanovic et al., “Could TCR Antagonism Explain Associations Between MHC Genes and Disease?” Trends Mol Med 9:139-146 (2003), which is hereby incorporated by reference in its entirety). However, irrespective of the nature of HLA association with disease, the individuals carrying poor prognosis alleles for given diseases would also constitute candidates for “peripheral application” of high reactivity TCR repertoire in both CD4⁺ and CD8⁺ T cell subsets.

Because of an inherent reactivity of TCR with self peptide/MHC complexes, increasing affinity for foreign antigen/MHC complexes inevitably increases the affinity of the TCR for self peptide/MHC complexes (Holler et al., “TCRs with High Affinity for Foreign pMHC Show Self Reactivity,” Nature Immunol 4:55-62 (2003), which is hereby incorporated by reference in its entirety). This carries the risk of developing autoimmunity. Sometimes this autoimmunity is relatively benign and worth the risk, such as the development of vitiligo in melanoma patients (Overwijk et al., Tumor Regression and Autoimmunity After Reversal of a Functionally Tolerant State of Self-Reactive CD8⁺ Cells,” J Exp Med 198:569-580 (2003), which is hereby incorporated by reference in its entirety). However, autoimmune diseases can have much more serious consequences. Obvious advantages of the “peripheral” strategy are the immediate effects (T cells should be ready to respond to antigen immediately following injection) and development of desired specificity, and testing thereof, prior to injection of transduced T cells. Specificity could be geared towards self-MHC restricted, or allo-MHC restricted (Morris et al., “Prospects for Immunotherapy of Malignant Disease,” Clin Exp Immunol 131:1-7 (2003); Stauss, H., “Immunotherapy with CTLs Restricted by Nonself MHC,” Immunol Today 20:180-183 (1999), which are hereby incorporated by reference in their entirety). The latter would have to involve the use of donor peripheral blood leukocytes (PBLs), whereas the former allows both self and donor PBLs (albeit the self PBLs will lead to a cleaner system with less unexpected outcomes).

Another aspect of the present invention (referred to hereinafter as the “central” strategy) consists of transducing or transfecting bone marrow precursors with the nucleic acid molecule encoding the high sensitivity TCR β chain of the present invention. Both autologous and heterologous bone marrow can be used in this aspect of the present invention. The successful application of the invention using the central strategy for the treatment of infectious diseases and disregulated cell proliferation depends on the expression of the TCR β chain having higher sensitivity recognition of antigen than a wild type TCR in CD8⁺ T cells. Therefore, regulatory DNA sequences allowing specific expression in CD8⁺ cells (Kieffer et al., “Identification of a Candidate Regulatory Region in the Human CD8 Gene Complex by Colocalization of DNase I Hypersensitive Sites and Matrix Attachment Regions which Bind SATB1 and GATA-3,” J Immunol 168(8):3915-22 (2002), which is hereby incorporated by reference in its entirety) will be used in constructs for transfecting host cells.

In the “central” strategy, the transgenic cells are bone marrow precursors, therefore, they will undergo thymic selection following introduction into the subject. The “central” strategy is technically more complex than the “peripheral” strategy (in which the cells do not undergo thymic selection, but migrate directly to peripheral lymphoid organs). Nevertheless, it has certain advantages. For example, the limits on the TCR affinity/avidity imposed by thymic selection represent an advantage since the danger of auto-immunity under these conditions would be limited. Because of the perpetual renewal of bone marrow precursors, the “central” strategy will likely have more permanent effect, whereas transduced peripheral T cells have limits on self expansion and survival, and senescence will inevitably be a limiting factor in the “peripheral” strategy. Another advantage of “central” strategy is the fact that pre-rearranged (transduced) TCR β chain and, to a lesser degree, TCR α chain, prevents expression of endogenous TCR β and TCR α chains, respectively (Borgulya et al., “Exclusion and Inclusion of α and β T Cell Receptor Alleles,” Cell 69:529-537 (1992), which is hereby incorporated by reference in its entirety). This process, known as allelic exclusion, occurs only during T cell development in the thymus, and does not operate in the peripheral T cells. The consequences of possible expression of endogenous TCR chains is competition for binding with transduced TCR chains. Ultimately, this results in the lower levels of TCR with desired specificity. This problem can be bypassed using engineered TCR chains that can only pair with transduced, engineered partner chains (Willemsen et al., “Genetic Engineering of T Cell Specificity for Immunotherapy of Cancer,” Hum Immunol 64:56-68 (2003), which is hereby incorporated by reference in its entirety). However, for this to happen, both TCR β and TCR α chains have to be engineered and applied, and, as discussed previously, the use of single entire TCR is very limited.

The present invention also relates to a method of treating a lymphocyte-mediated disease in a subject. This method involves providing a T cell receptor β chain having higher sensitivity recognition of antigen than does a wild type T cell receptor β chain and introducing the T cell receptor β chain having higher sensitivity recognition of antigen to a subject having the lymphocyte-mediated disease under conditions to treat the lymphocyte-mediated disease. Suitable cells for transfection in this aspect of the present invention include hematopoietic progenitor cells including, without limitation, bone marrow cells, isolated bone marrow stem cells, umbilical cord blood cells, and G-CSF-mobilized peripheral blood leukocytes. Suitable subjects are mammals, including, without limitation, humans. The method of introduction is bone marrow transplantation of transgenic cells having the high sensitivity TCR β chain nucleic acid molecule inserted (i.e., the “central” strategy) described above.

This aspect of the present invention takes advantage of the fact that a TCR repertoire with high reactivity to ligands (i.e., antigens) may have a role in immune responses completely different from that described herein above, thus providing another manner in which such a TCR repertoire can be applied. As discussed above, interaction between TCR and ligands with relatively high sensitivity during thymic selection can result in generation of CD4⁺CD25⁺ regulatory T cells (Apostolou et al., “Origin of Regulatory T Cells with Known Specificity for Antigen,” Nat Immunol 3:756-763 (2002); Jordan et al., “Thymic Selection of CD4⁺CD25⁺Regulatory T Cells Induced by an Agonist Self-Peptide,” Nature Immunol 2:301-306 (2001), which are hereby incorporated by reference in their entirety). The production of T regulatory cells, in particular, CD4⁺ T cells with high-sensitivity TCR repertoire, containing enhanced regulatory activity, can be useful for the treatment of lymphocyte-mediated diseases, including autoimmune diseases (von Herrath et al., “Antigen-Induced Regulatory T Cells in Autoimmunity,” Nat Rev Immunol 3:223-232 (2003), which is hereby incorporated by reference in its entirety), allergic reactions and asthma (Akbari et al., “Role of Regulatory T Cells in Allergy and Asthma,” Curr Opin Immunol 15:627-633 (2003), which is hereby incorporated by reference in its entirety), and graft rejection in transplantation (Wood et al., “Regulatory T Cells in Transplantation Tolerance,” Nat Rev Immunol 3:199-210 (2003), which is hereby incorporated by reference in its entirety).

The practical implication of this use of high sensitivity-based regulatory cells is that any manipulation of TCR repertoire has to occur prior to thymic selection. This means autologous bone marrow transplantation. Thus, in this aspect of the present invention, the high sensitivity TCR repertoire is introduced to a subject having a lymphocyte-mediated disease via the “central” method described above using regulatory DNA sequences driving expression in CD4⁺ cells (including CD4⁺CD8⁺ thymocytes), by transfecting hematopoietic cells with the nucleic acid molecule encoding a high sensitivity TCR β chain of the present invention and transplanting the transgenic cells into a subject having a lymphocyte-mediated disease. In this manner, the same TCR repertoire that is utilized to enhance immune responses for treatment of tumors and infectious diseases can be used to suppress immune responses for treatment of graft rejection and of autoimmune, immunopathological, and allergic diseases. This aspect of the present invention encompasses treatment of immunopathological diseases, including, without limitation, chronic hepatitis, cholecystitis, ulcerative colitis, post-vaccination sequelae (encephalopathy, encephalitis, eczema vaccinatum, vaccinia), post-streptococcal glomerulonephritis, subacute bacterial endocarditis, polyarteritis nodosa, mixed essential cryoglobulinemia, coeliac enteropathy, Crohn's disease, sarcoidosis, and aftous stomatitis. This method is also suitable for treatment of autoimmune diseases, including, without limitation, lupus, rheumatoid arthritis, spondylarthropathies, Sjogren's syndrome, polymyositis, scleroderma, dermatomyositis, multiple sclerosis, autoimmune polyneuritis, myasthenia gravis, Type 1 (juvenile) diabetes, insulin-resistant diabetes, hyperthyroidism (Graves' disease), autoimmune (Hashimoto's) thyroiditis, autoimmune adrenal insufficiency (Addison's disease), autoimmune oophoritis, autoimmune orchitis, autoimmune hemolytic anemia, paroxysmal cold hemoglobinuria, autoimmune thrombocytopenia, pernicious anemia, pure red cell anemia, autoimmune coagulopathies, pemphigus and other bullous diseases, psoriasis, rheumatic fever, vasculitis, Goodpasture's syndrome, postcardiotomy syndrome (Dressler's syndrome), billiary cirrhosis, and autoimmune hepatitis. This method is also suitable for allergic diseases, including, without limitation, asthma, atopic dermatitis, urticaria, serum sickness, drug allergy, insect allergy, anaphylaxis, food allergy, allergic gastroenteropathy, allergic rhinitis, allergic bronchopulmonary aspergillosis, hypersensitivity pneumonitis, and atopic keratoconjunctivitis.

The control over which function (enhanced effector function or regulatory function) will prevail can be maintained by the choice of cells used as recipients of TCR repertoire: the use of mature peripheral T cells will ensure effector function effects (via CD8⁺ T cells), whereas the use of bone marrow cells will allow the development of regulatory cells (via CD4⁺ T cells). Additionally, use of regulatory DNA sequences that drive CD4⁺ or CD8⁺ T cell-specific expression in making the high sensitivity TCR expression construct, as described herein above, can help manipulate the desired effects.

The present invention also relates to a transgenic mouse having a T cell receptor β chain having higher sensitivity recognition of antigen than a wild type mouse. This may be carried out using a method known as homologous recombination (Smithies et al., “Insertion of DNA Sequences into the Human Chromosomal β Globin Locus by Homologous Recombination,” Nature 359:696-699 (1985), which is hereby incorporated by reference in its entirety). This involves, generally, the transfer of recombinant genes into embryonic stem cells (ES) in culture and then into live animals to produce genetically modified transgenic animals expressing the desired protein. Briefly, a targeting vector containing the desired mutation is introduced into embryonic-derived stem (ES) cells. Examples of suitable vectors include, without limitation, (1) an insertion vector as described by Capecchi, M. R., “Altering the Genome by Homologous Recombination,” Science 244(4910):1288-92 (1989), which is hereby incorporated by reference in its entirety; (2) a vector based upon promoter trap, polyadenylation trap, “hit and run” or “tag-and-exchange” strategies, as described by Bradley et al., “Modifying the Mouse: Design and Desire,” Biotechnology 10:534-39 (1992), and Askew et al., “Site-Directed Point Mutations in Embryonic Stem Cells: a Gene Targeting Tag-and-Exchange Strategy,” Mol. Cell Biol., 13:4115-24 (1993) (which are hereby incorporated by reference in their entirety), or others known in the art. Screening procedures are then utilized to identify transformed ES cells in which the targeted event has occurred. An appropriate cell is then cloned and maintained as a pure population. The genetically modified ES cells are then used to generate mice by injection into blastocysts and the chimeric blastocysts are allowed to mature to term following transfer to a pseudopregnant foster mother (Gossler et al., “Transgenesis by Means of Blastocyst-Derived Embryonic Stem Cell Lines,” Proc Natl Acad Sci USA 83:9065-9069 (1986), which is hereby incorporated by reference in its entirety). Transgenic progeny are screened for the presence of the gene. Chimeric offspring heterozygous for the desired trait are mated to obtain homozygous individuals, and colonies characterized by expression of the transgene are established. This technique has often been used to develop lines of transgenic animals carrying a null allele at a specific locus (i.e., a “knockout” for the specific gene), where the absence of a gene product is the goal. However, it is also useful for altering the expression of endogenous proteins or introducing genes encoding proteins that differ from the wild type protein (Evans, M. J., “Potential for Genetic Manipulation of Mammals,” Mol Biol Med 6:557-565 (1989); Mansour, S. L., “Gene Targeting in Murine Embryonic Stem Cells: Introduction of Specific Alterations into the Mammalian Genome,” Genet Anal Tech Appl 7:219-227 (1990); Askew et al., “Site-Directed Point Mutations in Embryonic Stem Cells: a Gene Targeting Tag-and-Exchange Strategy,” Mol. Cell Biol., 13:4115-24 (1993), which are hereby incorporated by reference in their entirety).

In an alternative (and simpler) method, the DNA of choice is introduced directly into an embryo. The preparation of the transgenic mouse of the present invention using such a method is described in detail in the Examples, below. Briefly, a high affinity mouse TCR β chain having an amino acid sequence of SEQ ID NO: 2 was identified and isolated from a β2m deficient mouse, sequenced, cloned, and introduced into a mouse embryo by microinjection. The resulting transgenic mouse line, harboring the nucleic acid molecule of the present invention having a nucleotide sequence of SEQ ID NO: 1, is useful per se as a model for testing the immune response of an individual having a high affinity TCR β chain.

The present invention also relates to a second method of treating a disease caused by infectious agents or disregulated proliferation of a cell type in a subject. This method involves providing an isolated mouse T cell receptor β chain having higher sensitivity recognition of antigen than a wild type T cell receptor β chain, linking the mouse T cell receptor β chain having higher sensitivity recognition of antigen with a human T cell receptor a chain, and introducing the linked mouse T cell receptor β chain and human T cell receptor a chain to a subject having the disease under conditions to treat the disease. In one aspect of the present invention, this method is carried out by isolating the high sensitivity TCR β chain from the transgenic mouse of the present invention, and linking it with one or more suitable human T cell receptor a chains. If the mouse TCR β reacts with the human MHC molecules, mouse TCR β will be the primary candidate for immunotherapy. The question that will have to be addressed first, however, is whether mouse TCR β can pair with human TCR α chains. If the answer is yes, than mouse TCR β will be used. If not, “humanizing” the mouse TCR β is the method of choice. Humanization has been demonstrated to work for antibodies, as well as more recently for TCRs (Stanislawski et al., “Circumventing Tolerance to a Human MDM2-Derived Tumor Antigen by TCR Gene Transfer,” Nature Immunol 2:962-970 (2001), which is hereby incorporated by reference in its entirety).

Therefore, in another aspect of the present invention, the high sensitivity TCR β chain from the transgenic mouse is isolated, or prepared synthetically using methods well-known in the art to prepare a polypeptide from a known amino acid sequence, in this case, the amino acid corresponding to SEQ ID NO: 2, and then modified to provide a “humanized” form of the high sensitivity mouse TCR β chain. The basis of this modification process is substitution of a portion of the mouse polypeptide comprising the TCR β chain that does not promote binding to the ligand. Thus, a portion, or segment of the mouse TCR β chain, which includes a cytoplasmic, transmembrane, and constant region of the extracellular TCR domains, is substituted with the equivalent portion derived from the human TCR β chain polypeptide. Alternatively, the identification of the human TCR β chain that is the counterpart of the mouse TCR β chain described here can be carried out. This can be accomplished by an in vitro or by an in vivo selection under conditions limiting expression of MHC molecules. The in vitro selection methodology for both mouse and human thymocytes has recently been described using stromal support cells engineered to express Notch ligand molecules (Lehar et al., “T Cell Development in Culture,” Immunity 17:689-692 (2002), which is hereby incorporated by reference in its entirety). To adapt the system to this aspect of the present invention, it will be necessary to generate β2-microglobulin-deficient variants of the supporting stromal cell line.

An in vivo approach would involve xenografting human bone marrow into β2-microglobulin-deficient mouse that would be on the SCID or NOD/SCID background. This involves techniques that are known in the art and have been used successfully, for example, for in vivo analysis of normal and malignant human pluripotent hematopoietic stem cells (Greiner at al., “SCID Mouse Models of Human Stem Cell Engraftment,” Stem Cells 16:166-177 (1998), Lapidot et al., “Mechanism of Human Stem Cell Migration and Repopulation of NOD/SCID and β2 nmull NOD/SCID Mice: The Role of SDF-1/CXCR4 Interactions,” Ann NY Acad Sci 938:83-95 (2001), which are hereby incorporated by reference in their entirety) and the development of human leukemia models in mice (Lapidot et al., “Immune-Deficient SCID and NOD/SCID Mice Models as Functional Assays for Studying Normal and Malignant Human Hematopoiesis,” J Mol Med 75:664-673 (1997), which is hereby incorporated by reference in its entirety). In this manner, a human equivalent of the TCR β chain described in this application can be prepared, isolated, inserted into a suitable host using the procedures described above, and introduced to a subject directly by injection. All vectors, host cells, methods of preparation, methods of introduction, and diseases caused by infectious agents or disregulated proliferation of a cell type in a subject which can be treated in this aspect of the present invention are as described herein above.

Another aspect of the present invention relates to second method of treating a lymphocyte-mediated disease in a subject. This method involves providing an isolated mouse T cell receptor β chain having higher sensitivity recognition of antigen than a wild type T cell receptor β chain, linking the mouse T cell receptor β chain having higher sensitivity recognition of antigen with one or more human T cell receptor a chains, and introducing the linked mouse T cell receptor β chain and human T cell receptor α chain to a subject having the disease, under conditions to treat the disease. This method involves the central strategy for introduction of the modified T cell receptor β chain of the present invention into a subject. As described above, the high sensitivity mouse TCR β chain may be isolated from the transgenic mouse for use in this aspect of the present invention, and is used with or without modifications, depending upon the need to “humanize” the TCR β chain so that it is capable of combination with a human TCR α chain for introduction into a subject. In this aspect of the present invention, the high-reactivity TCR-repertoire is applied in a “central” manner. As described above, this strategy is particularly useful for treatment of lymphocyte-mediated disease, due to the fact that if expression is restricted to the CD4⁺ subset, the predicted dominant outcome is generation of regulatory T cells. The effect of regulatory cells is highly dependent on the ratio of regulatory to effector T cells and the conditions of application of TCR β described here for the purposes of immunosuppression will have to be worked out in great detail in the future. Nevertheless, it appears clear even at this stage that organ transplantation, chronic inflammatory diseases of immune or autoimmune nature, and allergic diseases will constitute conditions where enhanced regulatory cell development brought about high affinity/avidity with peptide/MHC molecules will be sought. Thus, this aspect is useful for the treatment of all the lymphocyte-mediate diseases described above.

To direct the TCR specificity towards antigens of interest the transduction/injection procedure can be complemented with in vivo or in vitro stimulation with antigen. The former can be achieved with any accepted form of vaccination, while the latter can be achieved with any form of accepted in vitro expansion of T cells.

All aspects of the present invention can be achieved by direct introduction into the subject of the DNA (gene therapy) coding for an isolated mouse T cell receptor β chain having higher sensitivity recognition of antigen than a wild type T cell receptor β chain, linking the mouse T cell receptor β chain with a human T cell receptor β chain. The targeting of TCR β expression to various stages of T cell development can be achieved through the use of different DNA regulatory sequences (promoter/enhancer/silencer elements) and, consequently, all of the aspects of indirect introduction can theoretically be accomplished in a gene therapy approach (i.e., the central strategy described herein) as well. For example, one aspect of the present invention relates to production of a CD8⁺ high sensitivity TCR repertoire, suitable for treatment of a disease caused by infectious agents and for diseases, including cancer, caused by disregulated proliferation of a cell type in a subject. Thus, in this aspect of the present invention, the expression vector containing the nucleic acid molecule encoding the high sensitivity TCR β chain of the present invention is operably linked to regulatory DNA sequences that will drive the production and expression of CD8⁺ T cells in the transgenic host (Kieffer et al., “Identification of a Candidate Regulatory Region in the Human CD8 Gene Complex by Colocalization of DNase I Hypersensitive Sites and Matrix Attachment Regions which Bind SATB1 and GATA-3,” J Immunol 168(8):3915-22 (2002), which is hereby incorporated by reference in its entirety). Alternatively, in those aspects of the present invention that relate to the production of CD4⁺ regulatory T cells, suitable DNA regulatory sequences are those that will preferentially drive the production and expression of the invention in CD4⁺ T cells of the transgenic host (Marodon et al., “Specific Transgene Expression in Human and Mouse CD4⁺ Cells Using Lentiviral Vectors with Regulatory Sequences from the CD4 Gene,” Blood 101(9):3416-23 (2003), which is hereby incorporated by reference in its entirety). Because of the still apparent technical limitations and potential dangers of gene therapy, today the preferred choice would be the indirect method of transfecting host cells in vitro and injecting the transfected cells into subject. Nevertheless, as the research on technical application of gene therapy develops, gene therapy may become the primary choice.

Therefore, as described above, the present invention involves two strategies for the expression of the nucleic acid molecule encoding the high sensitivity TCR β chain of the present invention, and two methods of introducing the nucleic acid molecule into a selected subject. The “central” strategy is defined by maturation of transduced T cells in the thymus and involves transducing bone marrow precursors that will undergo thymic selection. This provides a renewable, and thus, more permanent population of T cells harboring the nucleic acid molecule encoding the high sensitivity TCR β chain of the present invention. The “peripheral” strategy of expression involves transducing PBLs, a source of peripheral T cells, which provides an immediate population of circulating T cells having the nucleic acid molecule of the present invention, but a population that will not undergo thymic selection, and will eventually be exhausted.

The introduction of the nucleic acid molecule of the present invention can be carried out in two ways for each of the above-described strategies. One method of gene introduction is the “direct” method, which is carried out by in vivo introduction of the nucleic acid molecule of the present invention into the subject's bone marrow by any safe and effective means used in the art for gene therapy. In this aspect, the nucleic acid molecule is prepared in a vector selected for its suitability for direct application, e.g., a viral vector. The second method of introducing the nucleic acid molecule of the present invention is the “indirect” method, in which PBLs or bone marrow-derived cells are transformed or transduced in vitro with the nucleic acid molecule encoding the high sensitivity TCR β chain of the present invention, and reintroduced to the subject. One skilled in the art will understand that the selection of the expression strategy and the selection of DNA regulatory sequences for vector preparation, in combination, determines which disease condition(s) are suitable for treatment. A comparison of the direct (in vivo) and indirect (in vitro cell transfection) methods of gene introduction, in combination with the central versus peripheral expression strategies and the disease conditions they are suitable for, are summarized in Table 1, below.

TABLE 1

| | | | Development | |
|---|---|---|---|---|
| | | | of CD4⁺- | |
| | | | CD25⁺ | |
| Method of | | Regulatory | Regulatory | |
| Nucleic Acid | | DNA | Cells | |
| Molecule | Initial | Sequences | (Central vs. | |
| Introduction | Transgene | Allowing | Peripheral | Diseases |
| Into Cells | Expression | Expression in | Strategy) | Treated |
| Direct (gene | Peripheral T | CD4⁺, CD8⁺ or | No | Infectious |
| therapy | cells | both subsets | (Peripheral | Diseases, |
| method) | | | strategy) | Cancer |
| | Bone | CD4⁺CD8⁺ | No | Infectious |
| | marrow cells | thymocytes and | (Central | Diseases, |
| | or most | CD8⁺ T cells | strategy) | Cancer |
| | immature | | | |
| | thymocytes | | | |
| | Bone | CD4⁺CD8⁺ | Yes | Auto- |
| | marrow cells | thymocytes and | (Central | immune, |
| | or most | CD4⁺ T cells | strategy) | Immuno- |
| | immature | | | pathology, |
| | thymocytes | | | Allergic, |
| | | | | Diseases, |
| | | | | Graft |
| | | | | Rejection |
| Indirect (in | Peripheral | CD4⁺, CD8⁺ | No | Infectious |
| vitro cell | T cells | or both subsets | (Peripheral | Diseases, |
| transfection | | | strategy) | Cancer |
| method) | | | | |
| | Bone | CD4⁺CD8⁺ | No | Infectious |
| | marrow cells | thymocytes and | (Central | Diseases, |
| | or equivalent | CD8⁺ T cells | strategy) | Cancer |
| | Bone | CD4⁺CD8⁺ | Yes | Auto- |
| | marrow cells | thymocytes and | (Central | immune, |
| | or equivalent | CD4⁺ T cells | strategy) | Immuno- |
| | | | | pathology, |
| | | | | Allergic |
| | | | | Diseases, |
| | | | | Graft |
| | | | | Rejection |

### EXAMPLES

### Example 1

### Mice and In Vivo Manipulations

β2m−/− or β2 m+/+ mice on C57B1/6 background were all purchased from Taconic Farms (Germantown, N.Y.). Mice were immunized by intravenous injection of 1×10⁷ irradiated RMA-S-L^{d} cells. RMA is an MHC class I-containing subline of the Rauscher virus-induced B6 lymphoma RBL-5. RMA-S is a TAP2-deficient tumor cell line variant of RMA (Sandberg et al., “Recognition of the Major Histocompatibility Complex Restriction Element Modulated CD8⁺ T cell Specificity and Compensates for Loss of T Cell Receptor Contacts with the Specific Peptides,” J Exp Med 189(6):883-893 (1999), which is hereby incorporated by reference in its entirety). In cells with mutant TAP genes, the MHC I molecules in the ER are unstable and are eventually translocated back into the cytosol, where they are degraded. Therefore, RMA-S is essentially MHC class I-deficient, albeit small amounts of MHC class I molecules can be found. Four weeks following the initial injections, mice were injected sub-cutaneously in the flank with 1×10⁶ RMA-S-L^{d} transfectants. Mice were scored for palpable tumor growth three times a week, and tumor dimensions were measured.

### Example 2

### Cells

The murine fibroblastoid cell line MC57G (H-2^{b} haplotype) was transfected with the PvuI-linearized pHbApr-1-neo expression vector containing H-2K^{d} or H-2L^{d} cDNAs using Lipofectin™ reagent (Life Technologies, Gaithersburg, Md.) according to the manufacturer's instructions. Transfectants were selected by 1 mg/ml G418, and screened by immunofluorescence, using SF1.1.1 (H-2K^{d}-specific) and B22 (H-2L^{d}-specific) monoclonal antibodies, and CTL assays using H-2K^{d}- and H-2L^{d}-restricted CTLs.

### Example 3

### RT-PCR Amplification and DNA Sequencing

Total mRNA was isolated from 1×10⁶ cells of alloreactive CD8⁺ T cell lines (Nesic et al., “Factors Influencing the Patterns of T Lymphocyte Allorecognition,” Transplant 73:797-803 (2002), which is hereby incorporated by reference in its entirety) using TRIzol™ Reagent (Life Technologies, Gaithersburg, Md.) according to the manufacturer's recommendations. The first strand cDNA synthesis was performed using Super Script™ Preamplification System for First Strand cDNA Synthesis (Life Technologies, Gaithersburg, Md.). PCR was carried out using Taq Polymerase (Fisher Scientific, Fairlawn, N.J.) in buffer containing 20 mM Tris-Cl pH 8.4, 50 mM KCl, 1.5 mM MgCl₂, and 0.5 mM dNTP mix. Forty cycles were performed each consisting of 1 min at 94° C., 1 min at 48° C., and 1.5 min at 72° C. Consensus TCRβ chain primers, three sets of consensus primers for TCRα (Osman et al., “Characterization of the T Cell Receptor Repertoire Causing Collagen Arthritis in Mice,” J Exp Med 177:387-395 (1993), which is hereby incorporated by reference in its entirety), and β-actin primers (Nesic et al., “Factors Influencing the Patterns of T Lymphocyte Allorecognition,” Transplant 73:797-803 (2002), which is hereby incorporated by reference in its entirety) were added at 20 mg/ml. PCR amplification products were either sequenced directly using amplification primers, or were ligated into pGEM-T (ProMega, Madison, Wis.) or PCR-Script™ (Stratagene, La Jolla, Calif.) vector and individual clones were sequenced using M13 reverse or T7 primers. Sequencing was carried out using Taq Dye Deoxy Terminator Cycle Sequencing Kit (Perkin Elmer-Roche, Branchburg, N.J.) according to manufacturer's instructions, and an ABI 373A Automatic DNA Sequencer (Applied Biosystems Inc., Foster City, Calif.).

### Example 4

### TCR β Chain Cloning and Transgenic Mouse Construction

cDNA was produced with oligoDT using the Gibco BRL Superscript cDNA cloning kit, starting from T cell line MD6 mRNA. Using the genomic sequence of the TCRβ region (GenBank Accession No. AE000663, which is hereby incorporated by reference in its entirety) as a target, a BLAST2 search was conducted, using the program “bl2seq” on-line sequence homology program available at the National Cancer Biotechnology/National Institutes of Health website, for a region in the TCRβ locus that is identical to the fragment of Vβ2 identified for line MD6 (GenBank Accession No. AF034159, which is hereby incorporated by reference in its entirety). This provided the entire gene sequence, including flanking non-coding regions. Two primers were then designed in the non-coding regions of the TCR gene, as follows:

(SEQ ID NO: 3)
P1-) Cβ 5′-GCGCTCAGGATGCATAAAAG-3′
(SEQ ID NO: 4)
P2-) Vβ2 5′-CCCCACAGAGATAGAGAGAACCTG-3′

These primers were used to amplify by PCR the complete sequence of the TCRβ gene. PCR products were cloned in the TA cloning vector kit (Invitrogen, Carlsbad, Calif.), using the manufacturer's protocol. Ten bacterial clones were chosen for miniprep plasmid isolation. Plasmid DNA was tested for presence of the TCRβ insert by PCR using the above listed primers (SEQ ID NO: 3 and SEQ ID NO: 4). Positive plasmids were further checked for the presence of insert by digestion with EcoRI restriction enzyme. Two clones, arbitrarily called Vβ2 and Vβ6, were then sequenced to check for mutation using standard methods of the art described in Sambrook et al., Molecular Cloning: a Laboratory Manual, 2nd Edition, Cold Spring Harbor, N.Y., Cold Spring Harbor Laboratory Press, at 11.45 (1989), which is hereby incorporated by reference in its entirety. Sequencing was repeated until all base calls could be assigned without ambiguity (2-3×).

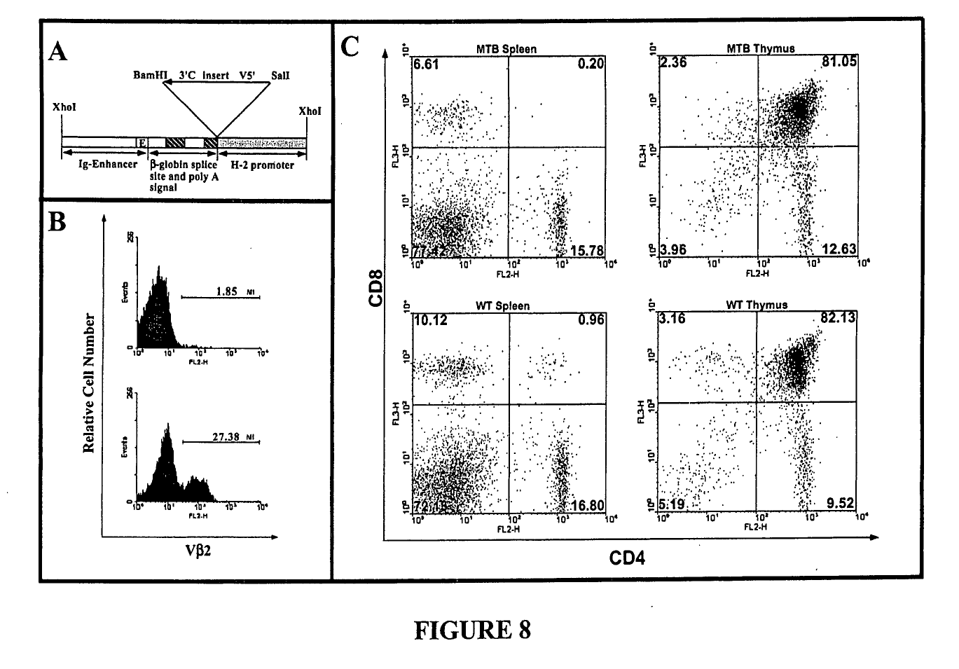

The plasmid pHSE3′ (Pircher et al., “Viral Escape by Selection of Cytotoxic T Cell-Resistant Virus Variants in vivo,” Nature 346:629-633(1990), which is hereby incorporated by reference in its entirety) was then treated with the restriction enzyme SalI and the opened fragment treated with the Klenow fragment of E. coli polymerase I to fill in nucleotides and produce a “blunt end” for ligation reaction. The plasmid was further digested with BamHI restriction enzyme. After the second digest the plasmid was dephosphorylated using calf intestinal phosphatase. The insert was prepared by direct digestion from TA cloning vector using the restriction enzymes BamHI and EcoRV. Insert was purified from 0.8% agarose gel using a Qiagen kit (Chatsworth, Calif.). The insert was ligated to the open plasmid using T4 DNA ligase (Promega, Madison, Wis.) at 16° C. overnight. The ligation product was used to transform DH5α bacterial hosts. DH5α colonies were screened for the presence of plasmid by PCR and restriction digests with (BamHI-NdeI). TCR clones used for the transgenic experiment were sequenced again to eliminate any possibility of mutations. One clone (Vβ2-2) derived from clone Vβ2 of the first round of selection was used for large scale plasmid preparation. Plasmid was digested with the enzyme XhoI and a fragment of 2.6 kb was purified twice on 0.8% agarose gel. The insert was purified to sequencing grade using a Qiagen kit (Valencia, Calif.). The final product was used for microinjection experiments in mouse egg cells (Brown and et al., “Oocyte Injection in the Mouse,” Methods Mol Biol 180:39-70 (2002), which is hereby incorporated by reference in its entirety). Transgenic progeny were initially screened using the same primers used to identify the original insert using the above listed primers (SEQ ID NO: 3 and SEQ ID NO: 4). Later, screening was performed using FITC labeled Vβ2 Mab (Pharmingen, San Diego, Calif.) in combination with anti-mouse CD4 (Pharmingen, San Diego, Calif.), as described below in Example 8 (FIG. 8B).

### Example 5

### Proposed Experimental Strategy

The affinity of a TCR for peptide/MHC complex depends on the number of non-covalent bonds established between the TCR and a peptide/MHC complex. TCR makes contacts with the peptide held in the groove of MHC molecule and with the α helices of the MHC molecule itself. During selection of TCR repertoire in normal (wild type) conditions, TCRs are exposed to the total of ˜1-2×10⁵ MHC molecules per cell. The normal peptide diversity is 1×10³-1×10⁴, providing an average of 10-100 copies of individual peptide/MHC complexes, which is in the range of copies required for T cells to be activated (Christinck et al., “Peptide Binding to Class I MHC on Living Cells and Quantitation of Complexes Required for CTL Lysis,” Nature 352:67-70 (1991), which is hereby incorporated by reference in its entirety). The exact number of copies required for thymic selection is not known at the moment. However, it can only be greater, since the affinity/avidity of TCR for self-peptide/MHC is lower than for the antigen (Alam et al., “T-Cell Receptor Affinity and Thymocyte Positive Selection,” Nature 381:616-620 (1996), which is hereby incorporated by reference in its entirety). It is known that selection process involves interaction with specific, rare peptides (Santori et al., “Rare, Structurally Homologous Self-Peptides Promote Thymocyte Positive Selection,” Immunity 17:131-142 (2002), which is hereby incorporated by reference in its entirety). If the number of MHC molecules per cell is drastically reduced by a factor of 100 without affecting the diversity of self-peptides, then the average copy number of each individual peptide/MHC complex falls below one. This can be achieved, for example, by eliminating the light chain of MHC class I molecules-β2-microglobulin. The most abundant MHC class I allele in β2m-deficient mice is at the level of 5% of the wild type (Bix et al., “Functionally Conformed Free Class I Heavy Chains Exist on the Surface of β2 Microglobulin Negative Cells,” J Exp Med 176:829-834 (1992); Sandberg et al., “Recognition of the Major Histocompatibility Complex Restriction Element Modulates CD8⁺ T Cell Specificity and Compensates for Loss of T Cell Receptor Contacts With the Specific Peptide,” J Exp Med 189:883-893 (1999), which are hereby incorporated by reference in their entirety). Under these conditions, selection of TCRs based on specific interactions with rare self-peptide/MHC complexes becomes impossible. Indeed, CD8⁺ T cells are virtually missing from β2m-deficient mice (Koller et al., “Normal Development of Mice Deficient in β2M, MHC Class I Proteins, and CD8⁺ T Cells,” Science 248:1227-1230 (1990); Zijlstra et al., “β2-Microglobulin Deficient Mice Lack CD4-CD8⁺ Cytolytic T Cells,” Nature 344:742-746 (1990), which are hereby incorporated by reference in their entirety).

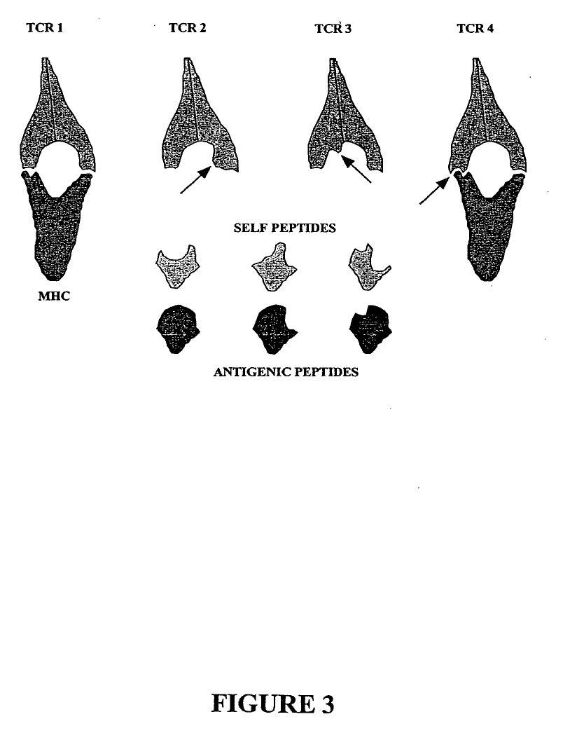

Some CD8⁺ T cells can be observed following immunization of β2m-deficient mice with either peptides (Cook et al., “Induction of Peptide-Specific CD8⁺ CTL Clones in β2-Microglobulin-Deficient Mice,” J Immunol 154:47-57 (1995), Sandberg et al., “Recognition of the Major Histocompatibility Complex Restriction Element Modulates CD8⁺ T Cell Specificity and Compensates for Loss of T Cell Receptor Contacts With the Specific Peptide,” J Exp Med 189:883-893 (1999), which are hereby incorporated by reference in their entirety) or alloantigens (Apasov et al., “Highly Lytic CD8⁺, αβ T Cell Receptor Cytotoxic T Cells with Major Histocompatibility Complex (MHC) Class I Antigen-Directed Cytotoxicity in β2-Microglobulin, MHC Class I-Deficient Mice,” Proc Natl Acad Sci USA 90:2837-2841 (1993); Apostolou et al., “Origin of Regulatory T Cells with Known Specificity for Antigen,” Nat Immunol 3:756-763 (2002); Glas et al., “The CD8⁺ T Cell Repertoire in β2-Microglobulin Deficient Mice is Biased Towards Reactivity Against Self-Major Histocompatibility Class I,” J Exp Med 179:661-672 (1994); Jhaver et al., “Apparent Split Tolerance of CD8⁺ T Cells From β2-Microglobulin-Deficient (β2m−/−) Mice to Syngeneic β2 m+/+ cells,” J Immunol 154:6252-6261 (1995), which are hereby incorporated by reference in their entirety). The appearance of these cells suggests the presence of rare, as determined by immunofluorescence, undetectable, CD8⁺ precursors that expand upon stimulation. An important characteristic of CD8⁺ T cells from β2m−/− mice is that they depend more on contacts with the MHC molecule and less on contacts with peptide, relative to wild type CD8⁺ T cells (Sandberg et al., “Recognition of the Major Histocompatibility Complex Restriction Element Modulates CD8⁺ T Cell Specificity and Compensates for Loss of T Cell Receptor Contacts With the Specific Peptide,” J Exp Med 189:883-893 (1999), which is hereby incorporated by reference in its entirety). Collectively, these findings suggest the following scenario. The thymic selection of the rare CD8⁺ T cells in β2m−/− mice must have followed a different pattern relative to the selection of majority of CD8⁺ T cells by wild type MHC class I. Because there is less than one copy, on average, of any single peptide-MHC class I complexes, the number of TCR interactions necessary for positive selection must have occurred by interactions of the TCR and MHC class I molecules occupied by different peptides, or even free of peptides. Unable to establish significant contacts with peptides due to many different peptide structures, these TCRs compensated by more contacts with MHC molecules. This outcome of this selection process is schematically represented in FIG. 3.

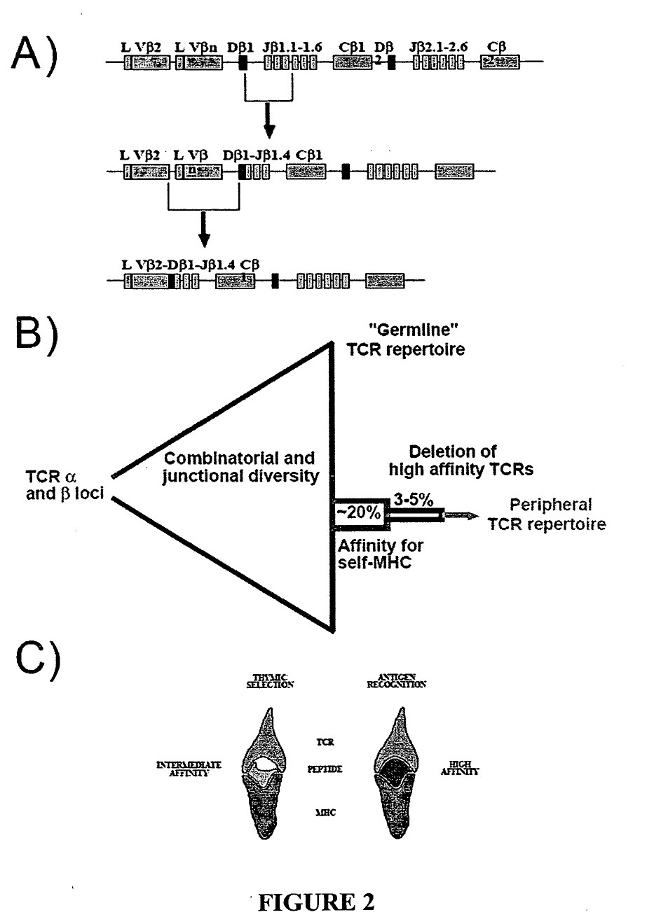

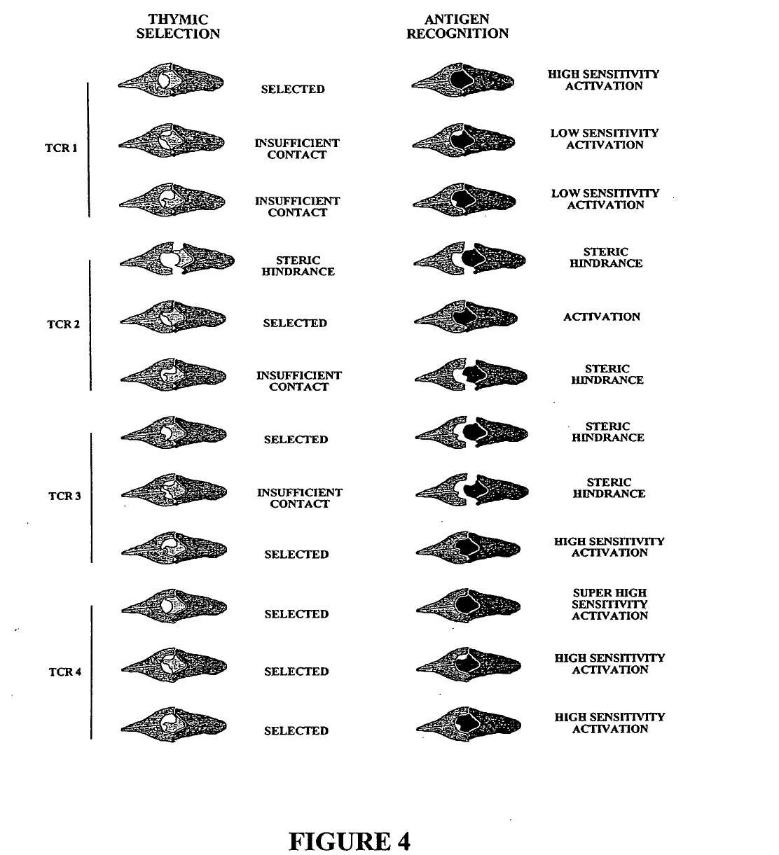

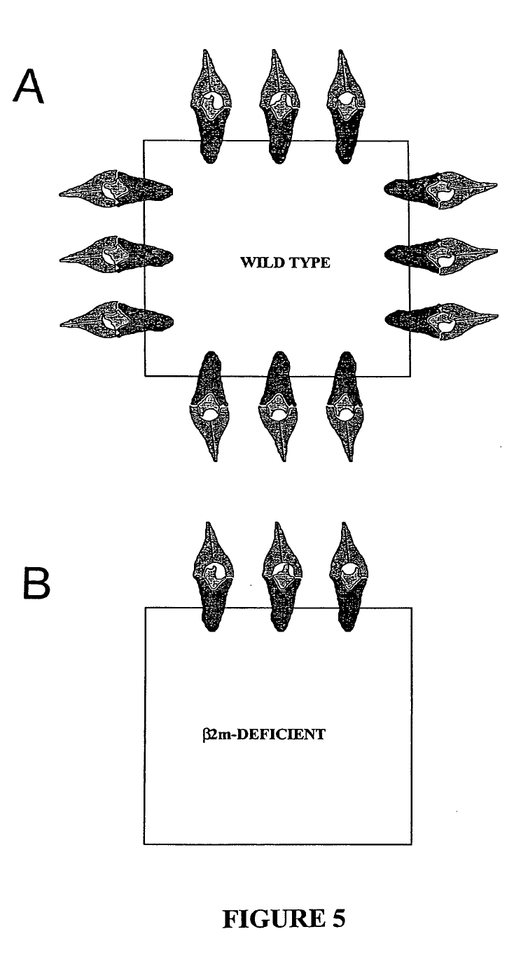

If one could isolate a TCR selected in β2m-deficient environment and force the expression of a part of the receptor responsible for enhanced interaction with the MHC molecule in the MHC class I wild type thymic environment while allowing random rearrangement of the other portion of TCR capable of interacting with both peptide and MHC, one would end up with a TCR repertoire characterized by overall significant increase in the number of contacts of TCRs with one component (MHC) and preserved or only marginally reduced number of contacts with the other component (peptide). This mode of TCR interaction with peptide/MHC should result in generally higher affinity of interaction, as shown in FIG. 4. Because one component of the TCR is fixed, the diversity of the germline TCR repertoire generated in this way will be reduced. Normally, reduced TCR diversity should result in inability to interact with some antigens. Nevertheless, for two reasons it is anticipated that this problem will be less significant than might be expected, or even non-existent. First, reduction of diversity will occur at the level of the germ-line TCR repertoire, which under normal conditions is subject to thymic selection that leaves only about 3-5% of the initial TCR repertoire, as shown in FIG. 2B. The majority of TCRs are eliminated because they cannot interact with the peptide/MHC complexes in the thymus (Surh et al., “T-Cell Apoptosis Detected in situ During Positive and Negative Selection in the Thymus,” Nature 372:100-103 (1994), which is hereby incorporated by reference in its entirety). Therefore, provision of a TCR chain capable of interacting with the peptide/MHC complexes will likely result in higher efficiency of selection and higher efficiency of interacting with different alleles of MHC. Second, the expected higher sensitivity of interaction with the antigens should enable the TCR repertoire to interact with antigens that wild type TCR repertoire is unable to react to. Thus, although the transgenic TCR repertoire may not be identical to the wild type (both in terms of expressed TCRs and array of recognized antigens), it is anticipated that TCR transgenic TCR repertoire should be functionally superior. FIG. 5A shows that a high density of MHC class I ligands in wild type cells allows the formation of copies of many diverse peptide/MHC complexes sufficient to allow individual peptide-based selection. FIG. 5B shows that MHC-based selection also operates, but diversity of this group of receptors is relatively low, and these receptors are normally difficult to detect. The low density of MHC class I in β2m-deficient thymus leads to formation of insufficient numbers of individual peptide/MHC complexes, and only selection of MHC-based TCRs is now allowed.

### Example 6

### TCR Diversity of MHC-Deficient Alloreactive CD8⁺ T cells

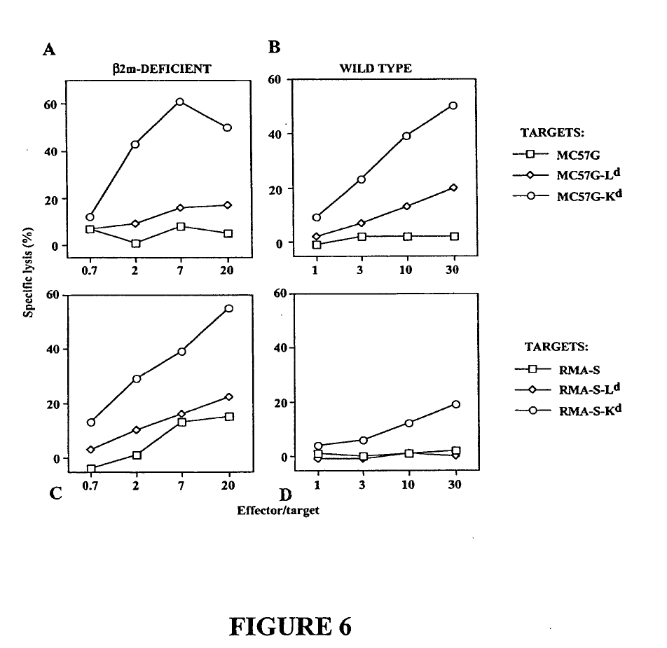

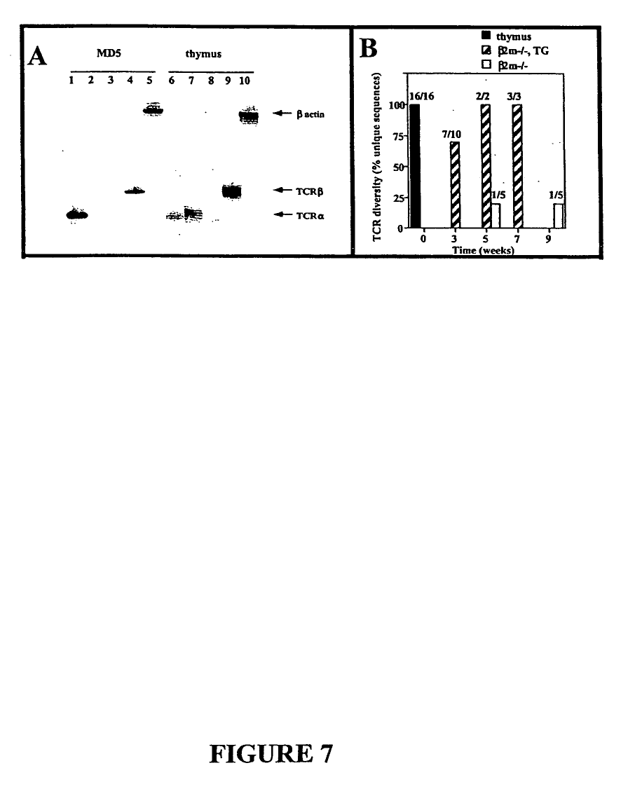

Alloreactive CD8⁺ T cells from β2m−/− mice recognize more efficiently than wild type CD8⁺ T cells target cells with reduced levels of alloantigens (Nesic et al., “Factors Influencing the Patterns of T Lymphocyte Allorecognition,” Transplant 73:797-803 (2002), which is hereby incorporated by reference in its entirety). FIGS. 6A-B show the allele-specificity of allo-reactive CD8⁺ T cell lines obtained from β2m−/− (line MD5) or wild type (line B6X) mice, respectively, tested in a chromium release assay. This reactivity most likely reflects the propensity of the TCRs isolated from β2m−/− mice to interact with the a helices of MHC molecules. An example of this reactivity is shown in FIGS. 6C-D, where β2m−/− CD8⁺ T cells lyse TAP-2-deficient RMA-S cells transfected with allogeneic MHC class I molecule significantly better than their wild type counterparts. To determine whether there may be a structural signature of enhanced allorecognition, CDR3 regions of TCR α and β in three β2m−/− CD8⁺ cell lines (designated MD5, MD6, and MD11) were analyzed (Nesic et al., “The Role of Protein Kinase C in CD8⁺ T Lymphocyte Effector Responses,” J Immunol 159:582-590 (1997), which is hereby incorporated by reference in its entirety). CDR3 regions were amplified by RT-PCR using consensus TCR β and TCR β primers (Osman et al., “Characterization of the T Cell Receptor Repertoire Causing Collagen Arthritis in Mice,” J Exp Med 177:387-395 (1993), which is hereby incorporated by reference in its entirety). CD8+ T cell lines are usually oligoclonal, and this might present difficulties in assigning the peptide-independent allorecognition to a particular CDR3 sequence. However, long in vitro culture of the cell lines usually results in drastic reduction of TCR complexity. To test whether the same might apply to the β2m−/− CD8⁺ cell lines of the present invention, the TCR usage was analyzed in MD5 cell line after 5 or 9 cycles of in vitro restimulation with alloantigen. Strikingly, PAGE analysis demonstrated discrete single bands for both TCR β and TCR β chains at both time points, in contrast to multiple bands that were obtained when cDNA from normal thymocytes was used as template, as shown in FIG. 7A. Single bands were also observed in the analysis of TCR expression in two other β2m−/− CD8⁺ cell lines. These findings suggest that the complexity of TCR usage in β2m−/− CD8⁺ lines may be limited. Sequencing of the cloned amplification products derived from MD5 cells and control cell lines (six alloreactive cell lines from β2m−/− mice grafted with β2 m+/+ thymus (Nesic et al., “The Role of Protein Kinase C in CD8⁺ T Lymphocyte Effector Responses,” J Immunol 159:582-590 (1997), which is hereby incorporated by reference in its entirety) further support this contention, shown in FIG. 7B. Collectively, these findings allow the tentative assignment of the functional properties of enhanced allorecognition to a given dominant TCR expressed by the MD5, MD6, or MD11 cell lines of 2m−/− origin, although presence of other TCRs cannot be formally excluded.

### Example 7

### WGG Motif Identified on CD8⁺ T Cells Originating from β2m−/−Mice

As shown in Table 2, below, sequences of TCR α and TCR β chains from MD5, MD6, and MD11 cell lines were next analyzed for the potential presence of common features. MD11 cells expressed a TCR composed of BV8.2, BD2.1, and BJ2.6 (Vβ chain) and AV8 and AV41 (Vα chain) elements, whereas MD5 and MD6 cells expressed identical TCR composed of BV2, BD2.1, BJ2.4 (Vβ chain), AV1, and AJ36 (Vα chain) elements. The fact that TCRs were identical in two lines originating from two separate β2m−/− mice speaks for the stringent selection of CD8⁺ TCR repertoire in β2m−/− background. Comparison of the two distinct CDR3 α and β chain sequences revealed a short shared sequence, or motif, (WGG) in the TCR β chains. This sequence was present in only one of the 12 TCRs isolated from CD8⁺ T cell lines originating from β2m−/− mice grafted with the β2 m+/+ thymus, shown in Table 3, below, and in none of the 8 TCRs isolated from β2 m+/+ CD8⁺ T cell lines or 16 TCRs from the β2 m+/+ thymus. No similarity was found in the amino acid sequences of CDR3 (joining) regions of TCR α chains isolated from the three β2m−/− CD8⁺ cell lines. Given that all three β2m−/− CD8⁺ cell lines recognize low levels of H-2K^{d} expressed by RMA-S cells (Nesic et al., “The Role of Protein Kinase C in CD8⁺ T Lymphocyte Effector Responses,” J Immunol 159:582-590 (1997), which is hereby incorporated by reference in its entirety), these results suggest that TCR β may determine allelic specificity and/or increased sensitivity during alloantigen recognition.

TABLE 2

| | | | | |
|---|---|---|---|---|
| CELL LINE | vα/vβ | V | CDR3 | C |

| | | | | |
|---|---|---|---|---|
| MD5, MD6 | vβ8 | SEQ ID NO:5 RTLYCTC . . . | SEQ ID NO:6 | SEQ ID NO:7 . . . FGAGTRLSVL |
| | | | . . . SAYWGGNTLY . . . | |
| MD5, MD6 | vα1 | SEQ ID NO:8 VSRPGSGG . . . | SEQ ID NO:9 | SEQ ID NO:10 . . . SRTQNLLCTS |
| | | | . . . KLTLGLEQDFRSTLT . . . | |
| MD11 | vβ8.2 | SEQ ID NO:11 SVYFCAS . . . | SEQ ID NO:12 | SEQ ID NO:13 . . . FGPGTRLTVL |
| | | | . . . GWGGSYEQY . . . | |
| MD11 | vα8 | SEQ ID NO:14 LSNWDN . . . | SEQ ID NO:15 | SEQ ID NO:16 . . . NIQNPEPAVY |
| | | | . . . TGYQNFYFGKGTSLTVIP . . . | |

TABLE 3

| CELL | | | | |
|---|---|---|---|---|
| LINE | Vαv | V | CDR3 | C |
| TS6 | vβ8.3 | SEQ. ID NO:17 SLYFCAS . . . | SEQ. ID NO:18 | SEQ. ID NO:19 . . . FGSGTRLLVI |
| | | | . . . SDGGNSDYT . . . | |
| TS6 | vβ8.2 | SEQ. ID NO:20 SVYFCAS . . . | SEQ. ID NO:21 | SEQ. ID NO:22 . . . FGPGTRLTVL |
| | | | . . . SDALFYEQY . . . | |
| TS6 | vβ8.2 | SEQ. ID NO:23 SVYFCAS . . . | SEQ. ID NO:24 | SEQ. ID NO:25 . . . FGHGTKLSVL |
| | | | . . . GDAQANERLF . . . | |
| TS7 | vβ8.2 | SEQ. ID NO:26 SVYFCAS . . . | SEQ. ID NO:27 | SEQ. ID NO:28 . . . FGSGTRLTVL |
| | | | . . . GDNSAETLY . . . | |
| TS7 | vβ2 | SEQ. ID NO:29 RTLYCTC . . . | SEQ. ID NO:30 | SEQ. ID NO:31 . . . FGAGTRLSVL |
| | | | . . . SAYWGGNTLY . . . | |
| TS8 | vβ8.2 | SEQ. ID NO:32 SVYFCAS . . . | SEQ. ID NO:33 | SEQ. ID NO:34 . . . FGPGTRLLVL |
| | | | . . . GDRGSQDTQY . . . | |
| TS8 | vβ13 | SEQ. ID NO:35 ATYLCAS . . . | SEQ. ID NO:36 | SEQ. ID NO:37 . . . FGPGTRLTVL |
| | | | . . .SPYEQY . . . | |
| TT2 | vβ8.3 | SEQ. ID NO:38 SLYFCAS . . . | SEQ. ID NO:39 | SEQ. ID NO:40 . . . FGAGTRLSVL |
| | | | . . . RDSQNTLY . . . | |
| TT2 | vβ8.2 | SEQ. ID NO:41 SVYFCAS . . . | SEQ. ID NO:42 | SEQ. ID NO:43 . . . FGPGTRLTVL |
| | | | . . . GEPLGAGEQY . . . | |
| TT2 | vβ8.3 | SEQ. ID NO:44 SLYFCAS . . . | SEQ. ID NO:45 | SEQ. ID NO:46 . . . FGSGTRLLVI |
| | | | . . . RSGTTNSDYT . . . | |
| TT3 | vβ8.2 | SEQ. ID NO:47 SVYFCAS . . . | SEQ. ID NO:48 | SEQ. ID NO:49 . . . FGKGTRLTVV |
| | | | . . . GANTEVF . . . | |
| TT3 | vβ2 | SEQ. ID NO:50 RTLYCTC . . . | SEQ. ID NO:51 | SEQ. ID NO:52 . . . FGPGTRLTVL |
| | | | . . . RAYYEQY . . . | |

### Example 8

### TCR β Chain from β2m−/− CD8⁺ Cells Confers Recognition of MHC Class I in TAP-Deficient Cells

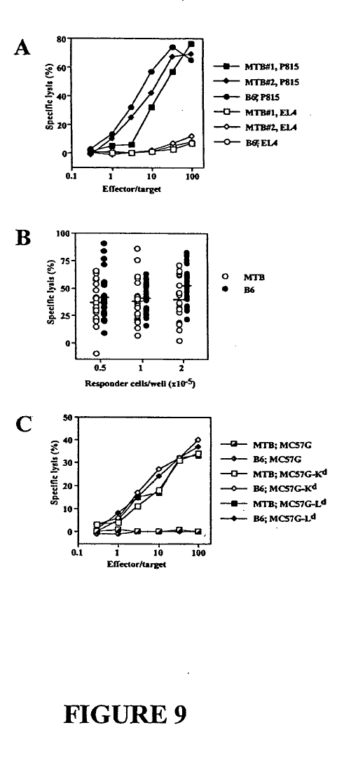

To determine the contribution of the TCR β chain to allorecognition, the entire TCR β chain from MD5 cells was cloned and placed under the control of the H-2 promoter and the Ig enhancer, shown in FIG. 8A. The expression of TCR β transgene in the transgenic mice is shown in FIG. 8B, lower panel. Transgenic mice generated using this construct (“MTB” mice) showed a normal number of T cells and a normal ratio of CD4⁺ and CD8⁺ T cells in both thymus and peripheral lymphoid tissues, shown in FIG. 8C. To test whether the transgenic TCR β chain could potentially contribute to increased levels of alloreactivity or could reproduce allele-specificity of the original TCR, wild type and transgenic spleen cells were cultured for five days with irradiated BALB/c (H-2^{d}) stimulator cells. Cytotoxic activity of MTB spleen cells against H-2^{d} target cells (P815) was not significantly different from that of their wild type littermates, as shown in FIG. 9A. H-2L^{d} was recognized equally well as H-2K^{d}, as seen in FIG. 9C, in contrast to the selective recognition of H-2K^{d} by the parental cell line, shown in FIG. 6. Differences in the overall level of alloreactivity were also not evident when responder cells were cultured under limiting dilution conditions, shown in FIG. 9B. If anything, frequency of alloreactive T cells in MTB mice was slightly lower. It would appear, therefore, that TCR β chain isolated from MD5 cells does not determine allele specificity and is not sufficient to confer intrinsic specificity for alloantigens.

### Example 9

### Selective Lysis by Transgenic MTB Cells Indicates Enhanced Antigen Recognition

To test whether transgenic TCR β chain may favor recognition of alloantigen with enhanced sensitivity, the lysis of TAP-2-mutant RMA-S cells transfected with H-2K^{d} or H-2L^{d} was determined. MTB effector cells stimulated with irradiated BALB/c splenocytes lysed RMA-S-K^{d} and RMA-S-L^{d} targets about three fold better than their littermate counterparts, shown in FIG. 10A. Better overall lysis of RMA-S-L^{d} targets reflects most likely higher expression of H-2L^{d} than that of H-2K^{d}. The difference in the lysis of RMA-S-L^{d} targets was much more pronounced when RMA-S-L^{d} cells were used as both stimulators and targets, shown in FIG. 10B. Interestingly, MTB CD8⁺ T cells stimulated with RMA-S-L^{d} cells were ineffective against the TAP-proficient P815 targets, as shown in FIG. 10D. Since TAP-proficient cells have even greater number of peptide-free MHC class I molecules at the cell surface than the TAP-deficient counterparts (Day et al., “Effect of TAP on the Generation and Intracellular Trafficking of Peptide-Receptive Major Histocompatibility Complex Class I Molecules,” Immunity 2:137-147 (1995), which is hereby incorporated by reference in its entirety), the most likely explanation for selective lysis of RMA-S cells is binding of the transgenic TCRs to a peptide that is presented in a TAP-independent manner and is competitively displaced by other peptides that require translocation by TAP. An alternative, but less likely, explanation would involve different conformation of peptide-free class I molecules presented by these two cell types.

If MTB TCR repertoire has enhanced sensitivity with MHC class I in general, then selection of CD8⁺ T cells in the TAP-deficient background should be more efficient than that of the wild type CD8⁺ T cells. Indeed, when MTB mice were bred to TAP-1-deficient background, CD8⁺ T cells were more abundant in both the thymus and the peripheral lymphoid tissues of MTB/TAP-1^{-/-} than of TAP-1^{-/-} control mice, as shown in FIGS. 11A-B. The numbers of CD8⁺ T cells in MTB/TAP-1^{-/-} mice were not completely restored to the wild type levels, suggesting that the frequency of TCR α chains involved in interaction with the MHC class I on the surface of TAP-1−/− cells is limited. Nevertheless, the impact of the transgenic TCR β is clear, as the same TCR α chains that selected CD8⁺ T cells in MTB/TAP-1^{-/-} mice were insufficient in the TAP1−/− background.

### Example 10

### Levels of CD5 Expression Elevated in MTB T Cells

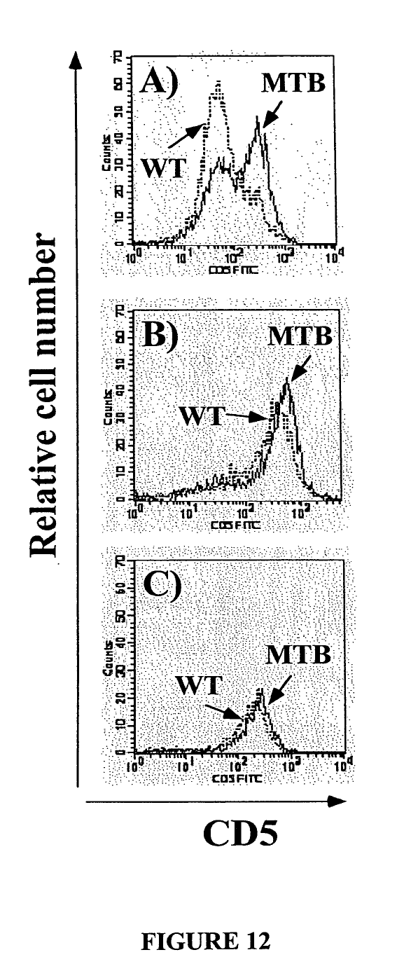

CD5 is a negative regulator of TCR signaling and the levels of CD5 expression is regulated by TCR signaling and TCR avidity for self-peptide/MHC complexes (Azzam et al., “CD5 Expression is Developmentally Regulated by T Cell Receptor (TCR) Signals and TCR Avidity,” J Exp Med 188:2301-2311 (1998); Wong et al., “Dynamic Tuning of T Cell Reactivity by Self-Peptide-Major Histocompatibility Complex Ligands,” J Exp Med 193:1179-1187 (2001), which are hereby incorporated by reference in their entirety). The first evidence of CD5 upregulation can be observed in CD4⁺CD8⁺ thymocytes before they undergo selection, where newly expressed TCRs sense their environment for strength of interactions with peptide/MHC complexes (Wong et al., “Dynamic Tuning of T Cell Reactivity by Self-Peptide-Major Histocompatibility Complex Ligands,” J Exp Med 193:1179-1187 (2001), which is hereby incorporated by reference in its entirety). The levels of CD5 are also maintained in the periphery with continuous engagement of TCR with self-peptide/MHC complexes (Smith et al., “Sensory Adaptation in Naive Peripheral CD4 T Cells,” J Exp Med 194:1253-1261 (2001), which is hereby incorporated by reference in its entirety). To determine the strength of interactions with self-peptide/MHC complexes perceived by MTB T cells, MTB and WT thymocytes, spleen, and lymph node cells were stained with anti-CD4, anti-CD8, and anti-CD5 monoclonal antibodies. Immunofluorescent analysis revealed significantly higher levels of CD5 in MTB double-positive (i.e., CD4+ CD8+) thymocytes, as shown in FIG. 12A. (The unlabeled peaks in FIG. 12A are likely the thymocytes that have not seen MHC, because they have not rearranged the TCRα chain.) In addition, CD5 levels were higher in MTB CD4⁺CD8- and CD4-CD8⁺ thymocytes, and CD4⁺ and CD8⁺ lymph node, as shown in FIG. 12B and FIG. 12C, respectively, and spleen cells, although the difference was not as dramatic as in CD4⁺CD8⁺ thymocytes. Therefore, it is concluded that signals from self-peptide/MHC complexes are perceived as relatively strong by most T cells expressing the transgenic TCRβ chain. Furthermore, the fact that levels of CD5 are elevated in both CD4⁺ and CD8⁺ T cells indicates that MTB TCRβ chain interacts very efficiently with MHC class II, as well as MHC class I.

Thus, a method to derive high affinity TCRs is provided herein that avoids directing antigen specificity and MHC restriction. Such a TCR repertoire is useful for treatment of infectious and proliferative diseases and would also provide a method directed to the treatment of autoimmune disease by enhancing the population of regulatory T cells in a subject.

### Example 11

### Transfer of MTB Spleen Cells Confers Immunity to TAP-Deficient Cells in Wild Type Mice

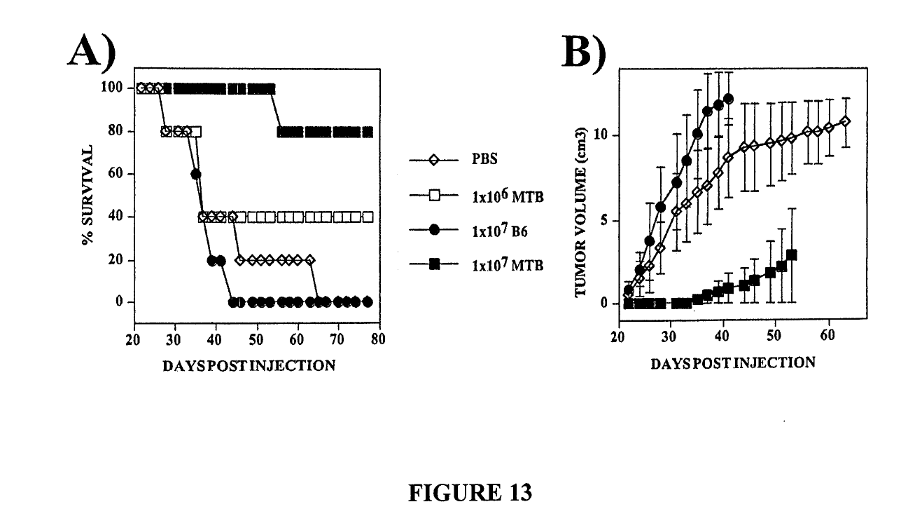

To test the in vivo effector activity of MTB cells, MTB or WT spleen cells were transferred to naive WT mice, and challenged with RMA-S-Ld live tumor cells. The growth of the injected tumors was followed every other day. Injection of 10×106 MTB cells significantly reduced the mortality, as shown in FIG. 13A, and the size of the tumor, as shown in FIG. 13B, relative to the recipients that received no spleen cells, while equal numbers of injected WT spleen cells were completely ineffective. Ten times smaller cell inoculum of MTB cells had a partial effect, as shown in FIGS. 13A-B. The protective effect of MTB cells is surprising, given that these cells were not previously primed with the tumor cells (neither in vivo in donors, nor in vitro prior to transfer). Therefore, it is concluded that the transgenic TCRβ chain confers a more effective immunity in vivo against the RMA-S-Ld tumor cells.

### Example 12

### Enhanced Regulatory Activity in MTB Mice

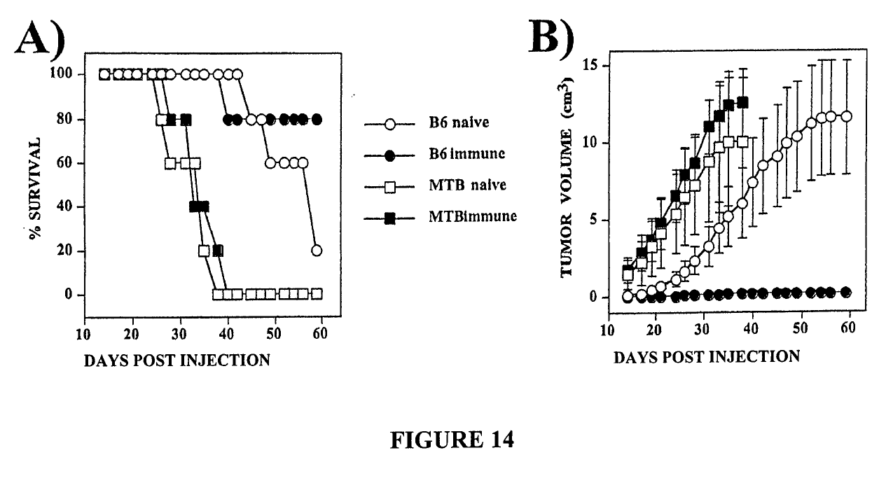

To determine whether MTB mice would exhibit increased levels of immunoregulatory activity, MTB and WT mice were injected with irradiated RMA-S-L^{d} cells. Four weeks later, these mice, as well as PBS-injected controls, were challenged with 1×10⁶ live RMA-S-L^{d} tumor cells. Comparison of tumor growth in immunized and PBS injected WT mice indicated the effect of prior immunization: most of the RMA-S-L^{d}-injected WT mice were able to control the growth of subsequently injected live tumor cells, whereas PBS-injected mice were not and had to be sacrificed, as shown in FIG. 14A. In contrast, MTB mice did not show the effect of immunization, as neither immunized nor PBS-injected mice were able to control the tumor growth, as shown in FIG. 14B. The inability of MTB mice to control the growth of RMA-S-L^{d} tumors in vivo is in stark contrast with their enhanced reactivity to the same cells in vitro and in vivo following transfer to naive recipients, as shown in FIGS. 13B. The dominance of effector over the regulatory MTB T cell function upon transfer is most likely related to the relative numbers of effector and regulatory cells injected into WT recipients and their potential to expand. It is therefore concluded that there are indications of both more efficient CD8⁺ T cell effector function and higher T cell regulatory activity in MTB mice in response to the challenge with live RMA-S-L^{d} tumor.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating a disease caused by infectious agents or disregulated proliferation of a cell type in a subject, said method comprising:
providing a T cell receptor β chain having higher sensitivity recognition of antigen than a wild type T cell receptor β chain and
introducing said T cell receptor β chain having higher sensitivity recognition of antigen to a subject having the disease under conditions effective to treat the disease.

2. The method according to claim 1, wherein said introducing comprises:
providing cells comprising the higher sensitivity recognition of antigen T cell receptor β chain and
injecting the cells into the subject.

3. The method according to claim 2, wherein the injected cells are peripheral T cells.

4. The method according to claim 2, wherein the cells are transfected with a nucleic acid molecule encoding the T cell receptor β chain.

5. The method according to claim 4, wherein the nucleic acid molecule comprises a nucleotide sequence of SEQ ID NO: 1.

6. The method according to claim 4, wherein the nucleic acid molecule encodes a protein comprising an amino acid sequence of SEQ ID NO: 2.

7. The method according to claim 4, wherein the nucleic acid molecule is under the control of DNA regulatory sequences driving CD8⁺ T-cell-specific expression.

8. The method according to claim 4, wherein the higher sensitivity of antigen recognition T cell receptor β chain comprises a CDR3 region having a WGG motif.

9. The method according to claim 1, wherein said introducing comprises:
providing a source of hematopoietic progenitor cells comprising a T cell receptor β chain having higher sensitivity recognition of antigen than does a wild type T cell receptor B chain and
transplanting the hematopoietic progenitor cells into the subject.

10. The method according to claim 9, wherein the source of hematopoietic progenitor cells is selected from the group consisting of bone marrow cells, isolated bone marrow stem cells, umbilical cord blood cells, and G-CSF-mobilized peripheral blood leukocytes.

11. The method according to claim 9, wherein the cells are transfected with a nucleic acid molecule encoding the higher sensitivity recognition of antigen T cell receptor β chain.

12. The method according to claim 11, wherein the nucleic acid molecule comprises a nucleotide sequence of SEQ ID NO: 1.

13. The method according to claim 11, wherein the nucleic acid molecule encodes a protein comprising an amino acid sequence of SEQ ID NO: 2.

14. The method according to claim 11, wherein the nucleic acid molecule is under the control of DNA regulatory sequences driving CD8⁺ T-cell-specific expression.

15. The method according to claim 9 further comprising:
allowing the transplanted cells to undergo thymic selection in the subject.

16. The method according to claim 1, wherein the disease is caused by infectious agents.

17. The method according to claim 16, wherein the disease caused by infectious agents is selected from the group consisting of AIDS, hepatitis A, B, C, cytomegalovirus, infectious mononucleosis, influenza, herpes, Varicella-zoster, yellow fever, dengue fever, smallpox, RSV, listeriosis, tuberculosis, leprosy, brucellosis, Legionnaire's disease, chlamydial infections, Rickettsial diseases, cholera, anthrax, Lyme disease, malaria, toxoplasmosis, giardiasis, trypanosomiasis, leishmaniasis, shistosomiasis, filariasis, candidiasis and other mycotic infections, cryptosporidium, microsporidium, histoplasma capsulatum, pneumocystis carinii, Cryptococcus neoformans, Coccidioides immitis, and helminic infections.

18. The method according to claim 1, wherein the disease is caused by disregulated proliferation of a cell type.

19. The method according to claim 18, wherein the disease caused by disregulated proliferation of a cell type is a cancer selected from the group consisting melanoma, breast cancer, prostate cancer, leukemia, lymphoma, mastocytoma, plasmocytoma, multiple myeloma, lung tumor, adenocarcinoma, ovarian cancer, testicular cancer, stomach cancer, intestinal cancer, neuroblastoma, pheochromocytoma, Wilms tumor, renal cell carcinoma, osteosarcoma, Ewing sarcoma, retinoblastoma, medulloblastoma, nasopharyngeal carcinoma, pancreatic carcinoma, hepatoblastoma, hepatoma, and cervical adenocarcinoma.

20. The method according to claim 1, wherein the subject is a mammal.

21. The method according to claim 20, wherein the mammal is a human.

22. A method of treating a lymphocyte-mediated disease in a subject, said method comprising:
providing a T cell receptor β chain having higher sensitivity recognition of antigen than does a wild type T cell receptor β chain and
introducing the T cell receptor β chain having higher sensitivity recognition of antigen to a subject having the lymphocyte-mediated disease under conditions effective to treat the lymphocyte-mediated disease.

23. The method according to claim 22, wherein said introducing comprises:
providing a source of hematopoietic progenitor cells comprising the T cell receptor β chain having higher sensitivity recognition of antigen than does a wild type and
transplanting the hematopoietic progenitor cells into the subject.

24. The method according to claim 23, wherein the source of hematopoietic progenitor cells is selected from the group consisting of bone marrow cells, isolated bone marrow stem cells, umbilical cord blood cells, and G-CSF-mobilized peripheral blood leukocytes.

25. The method according to claim 23, wherein the cells are transfected with a nucleic acid molecule encoding the T cell receptor β chain having higher sensitivity recognition of antigen.

26. The method according to claim 25, wherein the nucleic acid molecule comprises a nucleotide sequence of SEQ ID NO: 1.

27. The method according to claim 25, wherein the nucleic acid molecule encodes a protein comprising an amino acid sequence of SEQ ID NO: 2.

28. The method according to claim 25, wherein the higher sensitivity of antigen recognition T cell receptor β chain comprises a CDR3 region having a WGG motif.

29. The method according to claim 25, wherein the nucleic acid molecule is under the control of DNA regulatory sequences driving CD4⁺ T-cell-specific expression.

30. The method according to claim 23, further comprising:
allowing the transplanted cells to undergo thymic selection in the subject.

31. The method according to claim 22, wherein the lymphocyte-mediated disease is selected from the group consisting of an immunopathological disease, an autoimmune disease, an allergic disease, and organ or graft transplant rejection.

32. The method according to claim 31, wherein the lymphocyte-mediated disease is an immunopathological disease caused by an immune pathogen foreign to the subject or an undefined immunopathogen.

33. The method according to claim 32, wherein the immunopathological disease is selected from the group consisting of chronic hepatitis, cholecystitis, ulcerative colitis, post-vaccination sequelae, post-streptococcal glomerulonephritis, subacute bacterial endocarditis, polyarteritis nodosa, mixed essential cryoglobulinemia, coeliac enteropathy, Crohn's disease, sarcoidosis, and aftous stomatitis.

34. The method according to claim 31, wherein the lymphocyte-mediated disease is an autoimmune disease.

35. The method according to claim 34, wherein the autoimmune disease is selected from the group consisting of lupus, rheumatoid arthritis, spondylarthropathies, Sjogren's syndrome, polymyositis, scleroderma, dermatomyositis, multiple sclerosis, autoimmune polyneuritis, myasthenia gravis, Type 1 (juvenile) diabetes, insulin-resistant diabetes, hyperthyroidism (Graves' disease), autoimmune (Hashimoto's) thyroiditis, autoimmune adrenal insufficiency (Addison's disease), autoimmune oophoritis, autoimmune orchitis, autoimmune hemolytic anemia, paroxysmal cold hemoglobinuria, autoimmune thrombocytopenia, pernicious anemia, pure red cell anemia, autoimmune coagulopathies, pemphigus and other bullous diseases, psoriasis, rheumatic fever, vasculitis, Goodpasture's syndrome, postcardiotomy syndrome (Dressler's syndrome), billiary cirrhosis, and autoimmune hepatitis.

36. The method according to claim 31, wherein the lymphocyte-mediated disease is an allergic disease selected from the group consisting of asthma, atopic dermatitis, urticaria, serum sickness, drug allergy, insect allergy, anaphylaxis, food allergy, allergic gastroenteropathy, allergic rhinitis, allergic bronchopulmonary aspergillosis, hypersensitivity pneumonitis, and atopic keratoconjunctivitis.

37. The method according to claim 22, wherein the subject is a mammal.

38. The method according to claim 37, wherein the mammal is a human.

39. A transgenic mouse comprising:
a T cell receptor β chain having higher sensitivity recognition of antigen than a wild type mouse.

40. The transgenic mouse according to claim 39, wherein the T cell receptor β chain having higher sensitivity of recognition of antigen is encoded by a nucleic acid molecule comprising a nucleotide sequence of SEQ ID NO: 1.

41. The transgenic mouse according to claim 39, wherein the nucleic acid molecule encodes a T cell receptor β chain having higher sensitivity of recognition of antigen comprising an amino acid sequence of SEQ ID NO: 2.

42. The transgenic mouse according to claim 39, wherein the T cell receptor β chain having higher sensitivity recognition of antigen comprises a CDR3 region having a WGG motif.

43. A method of treating a disease caused by infectious agents or disregulated proliferation of a cell type in a subject, said method comprising:
providing an isolated mouse T cell receptor β chain having higher sensitivity recognition of antigen than a wild type T cell receptor β chain;
linking the mouse T cell receptor β chain with a human T cell receptor α chain, and
introducing said linked mouse T cell receptor β chain and human T cell receptor α chain to a subject having the disease under conditions effective to treat the disease.

44. The method according to claim 43, wherein the T cell receptor β chain is isolated from a transgenic mouse.

45. The method according to claim 43 further comprising:
modifying the mouse T cell receptor β chain to produce a T cell receptor β chain capable of linking with a human T cell receptor α chain.

46. The method according to claim 45, wherein said modifying comprises substituting one or more portions of a peptide comprising the mouse T cell receptor β chain with an equivalent portion derived from a human T cell receptor β chain polypeptide.

47. The method according to claim 46, wherein the portions of the human T cell receptor β chain polypeptide are selected from the group consisting of a cytoplasmic portion, a transmembrane portion, and a constant region of an extracellular domain.

48. The method according to claim 43, wherein said introducing comprises:
providing cells comprising the high sensitivity T cell receptor β chain and human T cell receptor α chain and
injecting the cells into the subject.

49. The method according to claim 48, wherein the injected cells are peripheral T cells.

50. The method according to claim 43, wherein the disease is caused by infectious agents.

51. The method according to claim 50, wherein the disease caused by infectious agents is selected from the group consisting of AIDS, hepatitis A, B, C, cytomegalovirus, infectious mononucleosis, influenza, herpes, Varicella-zoster, yellow fever, dengue fever, smallpox, RSV, listeriosis, tuberculosis, leprosy, brucellosis, Legionnaire's disease, Chlamydial infections, Rickettsial diseases, Cholera, Anthrax, Lyme disease, malaria, toxoplasmosis, giardiasis, trypanosomiasis, leishmaniasis, shistosomiasis, filariasis, candidiasis and other mycotic infections, cryptosporidium, microsporidium, histoplasma capsulatum, pneumocystis carinii, Cryptococcus neoformans, Coccidioides immitis, and helminic infections.

52. The method according to claim 43, wherein the disease is caused by disregulated proliferation of a cell type.

53. The method according to claim 52, wherein the disease caused by disregulated proliferation of a cell type is a cancer selected from the group consisting of melanoma, breast cancer, prostate cancer, leukemias, lymphomas, mastocytoma, plasmocytoma, multiple myeloma, lung tumor (adenocarcinoma), ovarian cancer, testicular cancer, stomach cancer, intestinal cancer, neuroblastoma, pheochromocytoma, Wilms tumor, renal cell carcinoma, osteosarcoma, Ewing sarcoma, retinoblastoma, medulloblastoma, nasopharyngeal carcinoma, pancreatic carcinoma, hepatoblastoma, hepatoma, and cervical adenocarcinoma.

54. The method according to claim 43, wherein the subject is a mammal.

55. The method according to claim 54, wherein the mammal is a human.

56. A method of treating a lymphocyte-mediated disease in a subject, said method comprising:
providing an isolated mouse T cell receptor β chain having higher sensitivity recognition of antigen than a wild type T cell receptor β chain;
linking the mouse T cell receptor β chain having higher sensitivity recognition of antigen with a human T cell receptor α chain, and
introducing said linked mouse T cell receptor β chain and human T cell receptor α chain to a subject having the lymphocyte-mediated disease under conditions effective to treat the disease.

57. The method according to claim 56, wherein the T cell receptor β chain is isolated from a transgenic mouse.

58. The method according to claim 56 further comprising:
modifying the mouse T cell receptor β chain to produce a T cell receptor β chain capable of linking with a human T cell receptor a chain.

59. The method according to claim 58, wherein said modifying comprises substituting one or more portions of a polypeptide comprising the mouse T cell receptor β chain with an equivalent portion derived from a human T cell receptor β chain polypeptide.

60. The method according to claim 59, wherein the portions of the human T cell receptor β chain polypeptide are selected from the group consisting of a cytoplasmic portion, a transmembrane portion, and a constant region of an extracellular domain.

61. The method according to claim 56, wherein said introducing comprises:
providing a source of hematopoietic progenitor cells comprising a T cell receptor β chain having higher sensitivity recognition of antigen than does a wild type T cell receptor β chain linked with a human T cell receptor a chain and
transplanting the cells into the subject.

62. The method according to claim 61, wherein the source of hematopoietic progenitor cells is selected from the group consisting of bone marrow cells, isolated bone marrow stem cells, umbilical cord blood cells, and G-CSF-mobilized peripheral blood leukocytes.

63. The method according to claim 61, wherein the cells are transfected with a nucleic acid molecule encoding the T cell receptor β chain having higher sensitivity recognition of antigen.

64. The method according to claim 63, wherein the nucleic acid molecule comprises a nucleotide sequence of SEQ ID NO: 1.

65. The method according to claim 63, wherein the higher sensitivity of antigen recognition T cell receptor β chain comprises a CDR3 region having a WGG motif.

66. The method according to claim 63, wherein the nucleic acid molecule encodes a protein comprising an amino acid sequence of SEQ ID NO: 2.

67. The method according to claim 61 further comprising:
allowing the transplanted cells to undergo thymic selection in the subject.

68. The method according to claim 56, wherein the lymphocyte-mediated disease is selected from the group consisting of an immunopathological disease, an autoimmune disease, an allergic disease, and organ or graft transplant rejection.

69. The method according to claim 68, wherein the lymphocyte-mediated disease is an immunopathological disease caused by an immune pathogen foreign to the subject or an undefined immunopathogen, or both.

70. The method according to claim 69, wherein the immunopathological disease is selected from the group consisting of chronic hepatitis, cholecystitis, ulcerative colitis, post-vaccination sequelae, post-streptococcal glomerulonephritis, subacute bacterial endocarditis, polyarteritis nodosa, mixed essential cryoglobulinemia, coeliac enteropathy, Crohn's disease, sarcoidosis, and aftous stomatitis.

71. The method according to claim 68, wherein the lymphocyte-mediated disease is an autoimmune disease selected from the group consisting of lupus, rheumatoid arthritis, spondylarthropathies, Sjogren's syndrome, polymyositis, scleroderma, dermatomyositis, multiple sclerosis, autoimmune polyneuritis, myasthenia gravis, Type 1 (juvenile) diabetes, insulin-resistant diabetes, hyperthyroidism (Graves' disease), autoimmune (Hashimoto's) thyroiditis, autoimmune adrenal insufficiency (Addison's disease), autoimmune oophoritis, autoimmune orchitis, autoimmune hemolytic anemia, paroxysmal cold hemoglobinuria, autoimmune thrombocytopenia, pernicious anemia, pure red cell anemia, autoimmune coagulopathies, pemphigus and other bullous diseases, psoriasis, rheumatic fever, vasculitis, Goodpasture's syndrome, postcardiotomy syndrome (Dressler's syndrome), billiary cirrhosis, and autoimmune hepatitis.

72. The method according to claim 68, wherein the lymphocyte-mediated disease is an allergic disease selected from the group consisting of asthma, atopic dermatitis, urticaria, serum sickness, drug allergies, insect allergies, anaphylaxis, food allergies, allergic gastroenteropathy, allergic rhinitis, allergic bronchopulmonary aspergillosis, hypersensitivity pneumonitis, and atopic keratoconjunctivitis.

73. The method according to claim 56, wherein the subject is a mammal.

74. The method according to claim 73, wherein the mammal is a human.
